SEQUENCE LISTING (20 sequences)

SEQ ID NO: 1 - protein, 1278 aa, Homo sapiens
MTARGLALGLLLLLLCPAQVFSQSCVWYGECGIAYGDKRYNCEYSGPPKPLPKDGYDLVQELCPGFFFGNVSLCCDVRQLQTLKDNLQLPLQFLSRCPSCFYNLLNLFCELTCSPRQSQFLNVTATEDYVDPVTNQTKTNVKELQYYVGQSFANAMYNACRDVEAPSSNDKALGLLCGKDADACNATNWIEYMFNKDNGQAPFTITPVFSDFPVHGMEPMNNATKGCDESVDEVTAPCSCQDCSIVCGPKPQPPPPPAPWTILGLDAMYVIMWITYMAFLLVFFGAFFAVWCYRKRYFVSEYTPIDSNIAFSVNASDKGEASCCDPVSAAFEGCLRRLFTRWGSFCVRNPGCVIFFSLVFITACSSGLVFVRVTTNPVDLWSAPSSQARLEKEYFDQHFGPFFRTEQLIIRAPLTDKHIYQPYPSGADVPFGPPLDIQILHQVLDLQIAIENITASYDNETVTLQDICLAPLSPYNTNCTILSVLNYFQNSHSVLDHKKGDDFFVYADYHTHFLYCVRAPASLNDTSLLHDPCLGTFGGPVFPWLVLGGYDDQNYNNATALVITFPVNNYYNDTEKLQRAQAWEKEFINFVKNYKNPNLTISFTAERSIEDELNRESDSDVFTVVISYAIMFLYISLALGHIKSCRRLLVDSKVSLGIAGILIVLSSVACSLGVFSYIGLPLTLIVIEVIPFLVLAVGVDNIFILVQAYQRDERLQGETLDQQLGRVLGEVAPSMFLSSFSETVAFFLGALSVMPAVHTFSLFAGLAVFIDFLLQITCFVSLLGLDIKRQEKNRLDIFCCVRGAEDGTSVQASESCLFRFFKNSYSPLLLKDWMRPIVIAIFVGVLSFSIAVLNKVDIGLDQSLSMPDDSYMVDYFKSISQYLHAGPPVYFVLEEGHDYTSSKGQNMVCGGMGCNNDSLVQQIFNAAQLDNYTRIGFAPSSWIDDYFDWVKPQSSCCRVDNITDQFCNASVVDPACVRCRPLTPEGKQRPQGGDFMRFLPMFLSDNPNPKCGKGGHAAYSSAVNILLGHGTRVGATYFMTYHTVLQTSADFIDALKKARLIASNVTETMGINGSAYRVFPYSVFYVFYEQYLTIIDDTIFNLGVSLGAIFLVTMVLLGCELWSAVIMCATIAMVLVNMFGVMWLWGISLNAVSLVNLVMSCGISVEFCSHITRAFTVSMKGSRVERAEEALAHMGSSVFSGITLTKFGGIVVLAFAKSQIFQIFYFRMYLAMVLLGATHGLIFLPVLLSYIGPSVNKAKSCATEERYKGTERERLLNF

SEQ ID NO: 2 - DNA, 3834 bp, Artificial Sequence
  NPC1 cDNA
atgaccgctcgcggcctggcccttggcctcctcctgctgctactgtgtccagcgcaggtgttttcacagtcctgtgtttggtatggagagtgtggaattgcatatggggacaagaggtacaattgcgaatattctggcccaccaaaaccattgccaaaggatggatatgacttagtgcaggaactctgtccaggattcttctttggcaatgtcagtctctgttgtgatgttcggcagcttcagacactaaaagacaacctgcagctgcctctacagtttctgtccagatgtccatcctgtttttataacctactgaacctgttttgtgagctgacatgtagccctcgacagagtcagtttttgaatgttacagctactgaagattatgttgatcctgttacaaaccagacgaaaacaaatgtgaaagagttacaatactacgtcggacagagttttgccaatgcaatgtacaatgcctgccgggatgtggaggccccctcaagtaatgacaaggccctgggactcctgtgtgggaaggacgctgacgcctgtaatgccaccaactggattgaatacatgttcaataaggacaatggacaggcaccttttaccatcactcctgtgttttcagattttccagtccatgggatggagcccatgaacaatgccaccaaaggctgtgacgagtctgtggatgaggtcacagcaccatgtagctgccaagactgctctattgtctgtggccccaagccccagcccccacctcctcctgctccctggacgatccttggcttggacgccatgtatgtcatcatgtggatcacctacatggcgtttttgcttgtgttttttggagcattttttgcagtgtggtgctacagaaaacggtattttgtctccgagtacactcccatcgatagcaatatagctttttctgttaatgcaagtgacaaaggagaggcgtcctgctgtgaccctgtcagcgcagcatttgagggctgcttgaggcggctgttcacacgctgggggtctttctgcgtccgaaaccctggctgtgtcattttcttctcgctggtcttcattactgcgtgttcgtcaggcctggtgtttgtccgggtcacaaccaatccagttgacctctggtcagcccccagcagccaggctcgcctggaaaaagagtactttgaccagcactttgggcctttcttccggacggagcagctcatcatccgggcccctctcactgacaaacacatttaccagccatacccttcgggagctgatgtaccctttggacctccgcttgacatacagatactgcaccaggttcttgacttacaaatagccatcgaaaacattactgcctcttatgacaatgagactgtgacacttcaagacatctgcttggcccctctttcaccgtataacacgaactgcaccattttgagtgtgttaaattacttccagaacagccattccgtgctggaccacaagaaaggggacgacttctttgtgtatgccgattaccacacgcactttctgtactgcgtacgggctcctgcctctctgaatgatacaagtttgctccatgacccttgtctgggtacgtttggtggaccagtgttcccgtggcttgtgttgggaggctatgatgatcaaaactacaataacgccactgcccttgtgattaccttccctgtcaataattactataatgatacagagaagctccagagggcccaggcctgggaaaaagagtttattaattttgtgaaaaactacaagaatcccaatctgaccatttccttcactgctgaacgaagtattgaagatgaactaaatcgtgaaagtgacagtgatgtcttcaccgttgtaattagctatgccatcatgtttctatatatttccctagccttggggcacatcaaaagctgtcgcaggcttctggtggattcgaaggtctcactaggcatcgcgggcatcttgatcgtgctgagctcggtggcttgctccttgggtgtcttcagctacattgggttgcccttgaccctcattgtgattgaagtcatcccgttcctggtgctggctgttggagtggacaacatcttcattctggtgcaggcctaccagagagatgaacgtcttcaaggggaaaccctggatcagcagctgggcagggtcctaggagaagtggctcccagtatgttcctgtcatccttttctgagactgtagcatttttcttaggagcattgtccgtgatgccagccgtgcacaccttctctctctttgcgggattggcagtcttcattgactttcttctgcagattacctgtttcgtgagtctcttggggttagacattaaacgtcaagagaaaaatcggctagacatcttttgctgtgtcagaggtgctgaagatggaacaagcgtccaggcctcagagagctgtttgtttcgcttcttcaaaaactcctattctccacttctgctaaaggactggatgagaccaattgtgatagcaatatttgtgggtgttctgtcattcagcatcgcagtcctgaacaaagtagatattggattggatcagtctctttcgatgccagatgactcctacatggtggattatttcaaatccatcagtcagtacctgcatgcgggtccgcctgtgtactttgtcctggaggaagggcacgactacacttcttccaaggggcagaacatggtgtgcggcggcatgggctgcaacaatgattccctggtgcagcagatatttaacgcggcgcagctggacaactatacccgaataggcttcgccccctcgtcctggatcgacgattatttcgactgggtgaagccacagtcgtcttgctgtcgagtggacaatatcactgaccagttctgcaatgcttcagtggttgaccctgcctgcgttcgctgcaggcctctgactccggaaggcaaacagaggcctcaggggggagacttcatgagattcctgcccatgttcctttcggataaccctaaccccaagtgtggcaaagggggacatgctgcctatagttctgcagttaacatcctccttggccatggcaccagggtcggagccacgtacttcatgacctaccacaccgtgctgcagacctctgctgactttattgacgctctgaagaaagcccgacttatagccagtaatgtcaccgaaaccatgggcattaacggcagtgcctaccgagtatttccttacagtgtgttttatgtcttctacgaacagtacctgaccatcattgacgacactatcttcaacctcggtgtgtccctgggcgcgatatttctggtgaccatggtcctcctgggctgtgagctctggtctgcagtcatcatgtgtgccaccatcgccatggtcttggtcaacatgtttggagttatgtggctctggggcatcagtctgaacgctgtatccttggtcaacctggtgatgagctgtggcatctccgtggagttctgcagccacataaccagagcgttcacggtgagcatgaaaggcagccgcgtggagcgcgcggaagaggcacttgcccacatgggcagctccgtgttcagtggaatcacacttacaaaatttggagggattgtggtgttggcttttgccaaatctcaaattttccagatattctacttcaggatgtatttggccatggtcttactgggagccactcacggattaatatttctccctgtcttactcagttacatagggccatcagtaaataaagccaaaagttgtgccactgaagagcgatacaaaggaacagagcgcgaacggcttctaaatttc

SEQ ID NO: 3 - DNA, 141 bp, Artificial Sequence
  5 prime Inverted Terminal Repeat
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 4 - DNA, 141 bp, Artificial Sequence
  3 prime Inverted Terminal Repeat
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 5 - DNA, 278 bp, Gallus gallus
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 6 - DNA, 240 bp, Artificial Sequence
  GUSB240 Promoter
cggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggccgagcgggggaccgggaagc

SEQ ID NO: 7 - DNA, 379 bp, Artificial Sequence
  GUSB379 Promoter
attcctgctgggaaaagcaagtggaggtgctccttgaagaaacagggggatcccaccgatctcaggggttctgttctggcctgcggccctggatcgtccagcctgggtcggggtggggagcagacctcgcccttatcggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggtcgagcgggggaccgggaagctta

SEQ ID NO: 8 - DNA, 146 bp, Unknown
  Herpes simplex virus
atgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaa

SEQ ID NO: 9 - DNA, 301 bp, Unknown
  Cytomegalovirus sp.
tacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 10 - DNA, 97 bp, Unknown
  simian virus 40
gtaagtttagtctttttgtcttttatttcaggtcccggatccggtggtggtgcaaatcaaagaactgctcctcagtggatgttgcctttacttctag

SEQ ID NO: 11 - DNA, 133 bp, Artificial Sequence
  Chimeric Intron
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 12 - DNA, 122 bp, Unknown
  simian virus 40
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 13 - DNA, 56 bp, Oryctolagus cuniculus
aataaaggaaatttattttcattgcaatagtgtgttggaattttttgtgtctctca

SEQ ID NO: 14 - DNA, 4685 bp, Artificial Sequence
  AAV transfer cassette 1
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgactcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgatgcatgtaagtttagtctttttgtcttttatttcaggtcccggatccggtggtggtgcaaatcaaagaactgctcctcagtggatgttgcctttacttctagcaccggtcgccaccatgaccgctcgcggcctggcccttggcctcctcctgctgctactgtgtccagcgcaggtgttttcacagtcctgtgtttggtatggagagtgtggaattgcatatggggacaagaggtacaattgcgaatattctggcccaccaaaaccattgccaaaggatggatatgacttagtgcaggaactctgtccaggattcttctttggcaatgtcagtctctgttgtgatgttcggcagcttcagacactaaaagacaacctgcagctgcctctacagtttctgtccagatgtccatcctgtttttataacctactgaacctgttttgtgagctgacatgtagccctcgacagagtcagtttttgaatgttacagctactgaagattatgttgatcctgttacaaaccagacgaaaacaaatgtgaaagagttacaatactacgtcggacagagttttgccaatgcaatgtacaatgcctgccgggatgtggaggccccctcaagtaatgacaaggccctgggactcctgtgtgggaaggacgctgacgcctgtaatgccaccaactggattgaatacatgttcaataaggacaatggacaggcaccttttaccatcactcctgtgttttcagattttccagtccatgggatggagcccatgaacaatgccaccaaaggctgtgacgagtctgtggatgaggtcacagcaccatgtagctgccaagactgctctattgtctgtggccccaagccccagcccccacctcctcctgctccctggacgatccttggcttggacgccatgtatgtcatcatgtggatcacctacatggcgtttttgcttgtgttttttggagcattttttgcagtgtggtgctacagaaaacggtattttgtctccgagtacactcccatcgatagcaatatagctttttctgttaatgcaagtgacaaaggagaggcgtcctgctgtgaccctgtcagcgcagcatttgagggctgcttgaggcggctgttcacacgctgggggtctttctgcgtccgaaaccctggctgtgtcattttcttctcgctggtcttcattactgcgtgttcgtcaggcctggtgtttgtccgggtcacaaccaatccagttgacctctggtcagcccccagcagccaggctcgcctggaaaaagagtactttgaccagcactttgggcctttcttccggacggagcagctcatcatccgggcccctctcactgacaaacacatttaccagccatacccttcgggagctgatgtaccctttggacctccgcttgacatacagatactgcaccaggttcttgacttacaaatagccatcgaaaacattactgcctcttatgacaatgagactgtgacacttcaagacatctgcttggcccctctttcaccgtataacacgaactgcaccattttgagtgtgttaaattacttccagaacagccattccgtgctggaccacaagaaaggggacgacttctttgtgtatgccgattaccacacgcactttctgtactgcgtacgggctcctgcctctctgaatgatacaagtttgctccatgacccttgtctgggtacgtttggtggaccagtgttcccgtggcttgtgttgggaggctatgatgatcaaaactacaataacgccactgcccttgtgattaccttccctgtcaataattactataatgatacagagaagctccagagggcccaggcctgggaaaaagagtttattaattttgtgaaaaactacaagaatcccaatctgaccatttccttcactgctgaacgaagtattgaagatgaactaaatcgtgaaagtgacagtgatgtcttcaccgttgtaattagctatgccatcatgtttctatatatttccctagccttggggcacatcaaaagctgtcgcaggcttctggtggattcgaaggtctcactaggcatcgcgggcatcttgatcgtgctgagctcggtggcttgctccttgggtgtcttcagctacattgggttgcccttgaccctcattgtgattgaagtcatcccgttcctggtgctggctgttggagtggacaacatcttcattctggtgcaggcctaccagagagatgaacgtcttcaaggggaaaccctggatcagcagctgggcagggtcctaggagaagtggctcccagtatgttcctgtcatccttttctgagactgtagcatttttcttaggagcattgtccgtgatgccagccgtgcacaccttctctctctttgcgggattggcagtcttcattgactttcttctgcagattacctgtttcgtgagtctcttggggttagacattaaacgtcaagagaaaaatcggctagacatcttttgctgtgtcagaggtgctgaagatggaacaagcgtccaggcctcagagagctgtttgtttcgcttcttcaaaaactcctattctccacttctgctaaaggactggatgagaccaattgtgatagcaatatttgtgggtgttctgtcattcagcatcgcagtcctgaacaaagtagatattggattggatcagtctctttcgatgccagatgactcctacatggtggattatttcaaatccatcagtcagtacctgcatgcgggtccgcctgtgtactttgtcctggaggaagggcacgactacacttcttccaaggggcagaacatggtgtgcggcggcatgggctgcaacaatgattccctggtgcagcagatatttaacgcggcgcagctggacaactatacccgaataggcttcgccccctcgtcctggatcgacgattatttcgactgggtgaagccacagtcgtcttgctgtcgagtggacaatatcactgaccagttctgcaatgcttcagtggttgaccctgcctgcgttcgctgcaggcctctgactccggaaggcaaacagaggcctcaggggggagacttcatgagattcctgcccatgttcctttcggataaccctaaccccaagtgtggcaaagggggacatgctgcctatagttctgcagttaacatcctccttggccatggcaccagggtcggagccacgtacttcatgacctaccacaccgtgctgcagacctctgctgactttattgacgctctgaagaaagcccgacttatagccagtaatgtcaccgaaaccatgggcattaacggcagtgcctaccgagtatttccttacagtgtgttttatgtcttctacgaacagtacctgaccatcattgacgacactatcttcaacctcggtgtgtccctgggcgcgatatttctggtgaccatggtcctcctgggctgtgagctctggtctgcagtcatcatgtgtgccaccatcgccatggtcttggtcaacatgtttggagttatgtggctctggggcatcagtctgaacgctgtatccttggtcaacctggtgatgagctgtggcatctccgtggagttctgcagccacataaccagagcgttcacggtgagcatgaaaggcagccgcgtggagcgcgcggaagaggcacttgcccacatgggcagctccgtgttcagtggaatcacacttacaaaatttggagggattgtggtgttggcttttgccaaatctcaaattttccagatattctacttcaggatgtatttggccatggtcttactgggagccactcacggattaatatttctccctgtcttactcagttacatagggccatcagtaaataaagccaaaagttgtgccactgaagagcgatacaaaggaacagagcgcgaacggcttctaaatttctaggtttaaacaagctttaagatacattgatgagtttggacaaaccacaactagaatgcagtgaaaaaaatgctttatttgtgaaatttgtgatgctattgctttatttgtaaccattataagctgcaataaacaagttctcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 15 - DNA, 4617 bp, Artificial Sequence
  AAV transfer cassette 2
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgaccggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatgcatgtaagtatcaaggttacaagacaggtttaaggagaccaatagaaactgggcttgtcgagacagagaagactcttgcgtttctgataggcacctattggtcttactgacatccactttgcctttctctccacagcaccggtcgccaccatgaccgctcgcggcctggcccttggcctcctcctgctgctactgtgtccagcgcaggtgttttcacagtcctgtgtttggtatggagagtgtggaattgcatatggggacaagaggtacaattgcgaatattctggcccaccaaaaccattgccaaaggatggatatgacttagtgcaggaactctgtccaggattcttctttggcaatgtcagtctctgttgtgatgttcggcagcttcagacactaaaagacaacctgcagctgcctctacagtttctgtccagatgtccatcctgtttttataacctactgaacctgttttgtgagctgacatgtagccctcgacagagtcagtttttgaatgttacagctactgaagattatgttgatcctgttacaaaccagacgaaaacaaatgtgaaagagttacaatactacgtcggacagagttttgccaatgcaatgtacaatgcctgccgggatgtggaggccccctcaagtaatgacaaggccctgggactcctgtgtgggaaggacgctgacgcctgtaatgccaccaactggattgaatacatgttcaataaggacaatggacaggcaccttttaccatcactcctgtgttttcagattttccagtccatgggatggagcccatgaacaatgccaccaaaggctgtgacgagtctgtggatgaggtcacagcaccatgtagctgccaagactgctctattgtctgtggccccaagccccagcccccacctcctcctgctccctggacgatccttggcttggacgccatgtatgtcatcatgtggatcacctacatggcgtttttgcttgtgttttttggagcattttttgcagtgtggtgctacagaaaacggtattttgtctccgagtacactcccatcgatagcaatatagctttttctgttaatgcaagtgacaaaggagaggcgtcctgctgtgaccctgtcagcgcagcatttgagggctgcttgaggcggctgttcacacgctgggggtctttctgcgtccgaaaccctggctgtgtcattttcttctcgctggtcttcattactgcgtgttcgtcaggcctggtgtttgtccgggtcacaaccaatccagttgacctctggtcagcccccagcagccaggctcgcctggaaaaagagtactttgaccagcactttgggcctttcttccggacggagcagctcatcatccgggcccctctcactgacaaacacatttaccagccatacccttcgggagctgatgtaccctttggacctccgcttgacatacagatactgcaccaggttcttgacttacaaatagccatcgaaaacattactgcctcttatgacaatgagactgtgacacttcaagacatctgcttggcccctctttcaccgtataacacgaactgcaccattttgagtgtgttaaattacttccagaacagccattccgtgctggaccacaagaaaggggacgacttctttgtgtatgccgattaccacacgcactttctgtactgcgtacgggctcctgcctctctgaatgatacaagtttgctccatgacccttgtctgggtacgtttggtggaccagtgttcccgtggcttgtgttgggaggctatgatgatcaaaactacaataacgccactgcccttgtgattaccttccctgtcaataattactataatgatacagagaagctccagagggcccaggcctgggaaaaagagtttattaattttgtgaaaaactacaagaatcccaatctgaccatttccttcactgctgaacgaagtattgaagatgaactaaatcgtgaaagtgacagtgatgtcttcaccgttgtaattagctatgccatcatgtttctatatatttccctagccttggggcacatcaaaagctgtcgcaggcttctggtggattcgaaggtctcactaggcatcgcgggcatcttgatcgtgctgagctcggtggcttgctccttgggtgtcttcagctacattgggttgcccttgaccctcattgtgattgaagtcatcccgttcctggtgctggctgttggagtggacaacatcttcattctggtgcaggcctaccagagagatgaacgtcttcaaggggaaaccctggatcagcagctgggcagggtcctaggagaagtggctcccagtatgttcctgtcatccttttctgagactgtagcatttttcttaggagcattgtccgtgatgccagccgtgcacaccttctctctctttgcgggattggcagtcttcattgactttcttctgcagattacctgtttcgtgagtctcttggggttagacattaaacgtcaagagaaaaatcggctagacatcttttgctgtgtcagaggtgctgaagatggaacaagcgtccaggcctcagagagctgtttgtttcgcttcttcaaaaactcctattctccacttctgctaaaggactggatgagaccaattgtgatagcaatatttgtgggtgttctgtcattcagcatcgcagtcctgaacaaagtagatattggattggatcagtctctttcgatgccagatgactcctacatggtggattatttcaaatccatcagtcagtacctgcatgcgggtccgcctgtgtactttgtcctggaggaagggcacgactacacttcttccaaggggcagaacatggtgtgcggcggcatgggctgcaacaatgattccctggtgcagcagatatttaacgcggcgcagctggacaactatacccgaataggcttcgccccctcgtcctggatcgacgattatttcgactgggtgaagccacagtcgtcttgctgtcgagtggacaatatcactgaccagttctgcaatgcttcagtggttgaccctgcctgcgttcgctgcaggcctctgactccggaaggcaaacagaggcctcaggggggagacttcatgagattcctgcccatgttcctttcggataaccctaaccccaagtgtggcaaagggggacatgctgcctatagttctgcagttaacatcctccttggccatggcaccagggtcggagccacgtacttcatgacctaccacaccgtgctgcagacctctgctgactttattgacgctctgaagaaagcccgacttatagccagtaatgtcaccgaaaccatgggcattaacggcagtgcctaccgagtatttccttacagtgtgttttatgtcttctacgaacagtacctgaccatcattgacgacactatcttcaacctcggtgtgtccctgggcgcgatatttctggtgaccatggtcctcctgggctgtgagctctggtctgcagtcatcatgtgtgccaccatcgccatggtcttggtcaacatgtttggagttatgtggctctggggcatcagtctgaacgctgtatccttggtcaacctggtgatgagctgtggcatctccgtggagttctgcagccacataaccagagcgttcacggtgagcatgaaaggcagccgcgtggagcgcgcggaagaggcacttgcccacatgggcagctccgtgttcagtggaatcacacttacaaaatttggagggattgtggtgttggcttttgccaaatctcaaattttccagatattctacttcaggatgtatttggccatggtcttactgggagccactcacggattaatatttctccctgtcttactcagttacatagggccatcagtaaataaagccaaaagttgtgccactgaagagcgatacaaaggaacagagcgcgaacggcttctaaatttctaggtttaaacaagcttaataaaggaaatttattttcattgcaatagtgtgttggaattttttgtgtctctcactcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 16 - DNA, 4720 bp, Artificial Sequence
  AAV Transfer Cassette 3
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacattcctgctgggaaaagcaagtggaggtgctccttgaagaaacagggggatcccaccgatctcaggggttctgttctggcctgcggccctggatcgtccagcctgggtcggggtggggagcagacctcgcccttatcggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggtcgagcgggggaccgggaagcttaatgcatgtaagtttagtctttttgtcttttatttcaggtcccggatccggtggtggtgcaaatcaaagaactgctcctcagtggatgttgcctttacttctagcaccggtcgccaccatgaccgctcgcggcctggcccttggcctcctcctgctgctactgtgtccagcgcaggtgttttcacagtcctgtgtttggtatggagagtgtggaattgcatatggggacaagaggtacaattgcgaatattctggcccaccaaaaccattgccaaaggatggatatgacttagtgcaggaactctgtccaggattcttctttggcaatgtcagtctctgttgtgatgttcggcagcttcagacactaaaagacaacctgcagctgcctctacagtttctgtccagatgtccatcctgtttttataacctactgaacctgttttgtgagctgacatgtagccctcgacagagtcagtttttgaatgttacagctactgaagattatgttgatcctgttacaaaccagacgaaaacaaatgtgaaagagttacaatactacgtcggacagagttttgccaatgcaatgtacaatgcctgccgggatgtggaggccccctcaagtaatgacaaggccctgggactcctgtgtgggaaggacgctgacgcctgtaatgccaccaactggattgaatacatgttcaataaggacaatggacaggcaccttttaccatcactcctgtgttttcagattttccagtccatgggatggagcccatgaacaatgccaccaaaggctgtgacgagtctgtggatgaggtcacagcaccatgtagctgccaagactgctctattgtctgtggccccaagccccagcccccacctcctcctgctccctggacgatccttggcttggacgccatgtatgtcatcatgtggatcacctacatggcgtttttgcttgtgttttttggagcattttttgcagtgtggtgctacagaaaacggtattttgtctccgagtacactcccatcgatagcaatatagctttttctgttaatgcaagtgacaaaggagaggcgtcctgctgtgaccctgtcagcgcagcatttgagggctgcttgaggcggctgttcacacgctgggggtctttctgcgtccgaaaccctggctgtgtcattttcttctcgctggtcttcattactgcgtgttcgtcaggcctggtgtttgtccgggtcacaaccaatccagttgacctctggtcagcccccagcagccaggctcgcctggaaaaagagtactttgaccagcactttgggcctttcttccggacggagcagctcatcatccgggcccctctcactgacaaacacatttaccagccatacccttcgggagctgatgtaccctttggacctccgcttgacatacagatactgcaccaggttcttgacttacaaatagccatcgaaaacattactgcctcttatgacaatgagactgtgacacttcaagacatctgcttggcccctctttcaccgtataacacgaactgcaccattttgagtgtgttaaattacttccagaacagccattccgtgctggaccacaagaaaggggacgacttctttgtgtatgccgattaccacacgcactttctgtactgcgtacgggctcctgcctctctgaatgatacaagtttgctccatgacccttgtctgggtacgtttggtggaccagtgttcccgtggcttgtgttgggaggctatgatgatcaaaactacaataacgccactgcccttgtgattaccttccctgtcaataattactataatgatacagagaagctccagagggcccaggcctgggaaaaagagtttattaattttgtgaaaaactacaagaatcccaatctgaccatttccttcactgctgaacgaagtattgaagatgaactaaatcgtgaaagtgacagtgatgtcttcaccgttgtaattagctatgccatcatgtttctatatatttccctagccttggggcacatcaaaagctgtcgcaggcttctggtggattcgaaggtctcactaggcatcgcgggcatcttgatcgtgctgagctcggtggcttgctccttgggtgtcttcagctacattgggttgcccttgaccctcattgtgattgaagtcatcccgttcctggtgctggctgttggagtggacaacatcttcattctggtgcaggcctaccagagagatgaacgtcttcaaggggaaaccctggatcagcagctgggcagggtcctaggagaagtggctcccagtatgttcctgtcatccttttctgagactgtagcatttttcttaggagcattgtccgtgatgccagccgtgcacaccttctctctctttgcgggattggcagtcttcattgactttcttctgcagattacctgtttcgtgagtctcttggggttagacattaaacgtcaagagaaaaatcggctagacatcttttgctgtgtcagaggtgctgaagatggaacaagcgtccaggcctcagagagctgtttgtttcgcttcttcaaaaactcctattctccacttctgctaaaggactggatgagaccaattgtgatagcaatatttgtgggtgttctgtcattcagcatcgcagtcctgaacaaagtagatattggattggatcagtctctttcgatgccagatgactcctacatggtggattatttcaaatccatcagtcagtacctgcatgcgggtccgcctgtgtactttgtcctggaggaagggcacgactacacttcttccaaggggcagaacatggtgtgcggcggcatgggctgcaacaatgattccctggtgcagcagatatttaacgcggcgcagctggacaactatacccgaataggcttcgccccctcgtcctggatcgacgattatttcgactgggtgaagccacagtcgtcttgctgtcgagtggacaatatcactgaccagttctgcaatgcttcagtggttgaccctgcctgcgttcgctgcaggcctctgactccggaaggcaaacagaggcctcaggggggagacttcatgagattcctgcccatgttcctttcggataaccctaaccccaagtgtggcaaagggggacatgctgcctatagttctgcagttaacatcctccttggccatggcaccagggtcggagccacgtacttcatgacctaccacaccgtgctgcagacctctgctgactttattgacgctctgaagaaagcccgacttatagccagtaatgtcaccgaaaccatgggcattaacggcagtgcctaccgagtatttccttacagtgtgttttatgtcttctacgaacagtacctgaccatcattgacgacactatcttcaacctcggtgtgtccctgggcgcgatatttctggtgaccatggtcctcctgggctgtgagctctggtctgcagtcatcatgtgtgccaccatcgccatggtcttggtcaacatgtttggagttatgtggctctggggcatcagtctgaacgctgtatccttggtcaacctggtgatgagctgtggcatctccgtggagttctgcagccacataaccagagcgttcacggtgagcatgaaaggcagccgcgtggagcgcgcggaagaggcacttgcccacatgggcagctccgtgttcagtggaatcacacttacaaaatttggagggattgtggtgttggcttttgccaaatctcaaattttccagatattctacttcaggatgtatttggccatggtcttactgggagccactcacggattaatatttctccctgtcttactcagttacatagggccatcagtaaataaagccaaaagttgtgccactgaagagcgatacaaaggaacagagcgcgaacggcttctaaatttctaggtttaaacaagcttaataaaggaaatttattttcattgcaatagtgtgttggaattttttgtgtctctcactcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 17 - DNA, 4683 bp, Artificial Sequence
  AAV transfer casette 4
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgaccggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatgcatgtaagtatcaaggttacaagacaggtttaaggagaccaatagaaactgggcttgtcgagacagagaagactcttgcgtttctgataggcacctattggtcttactgacatccactttgcctttctctccacagcaccggtcgccaccatgaccgctcgcggcctggcccttggcctcctcctgctgctactgtgtccagcgcaggtgttttcacagtcctgtgtttggtatggagagtgtggaattgcatatggggacaagaggtacaattgcgaatattctggcccaccaaaaccattgccaaaggatggatatgacttagtgcaggaactctgtccaggattcttctttggcaatgtcagtctctgttgtgatgttcggcagcttcagacactaaaagacaacctgcagctgcctctacagtttctgtccagatgtccatcctgtttttataacctactgaacctgttttgtgagctgacatgtagccctcgacagagtcagtttttgaatgttacagctactgaagattatgttgatcctgttacaaaccagacgaaaacaaatgtgaaagagttacaatactacgtcggacagagttttgccaatgcaatgtacaatgcctgccgggatgtggaggccccctcaagtaatgacaaggccctgggactcctgtgtgggaaggacgctgacgcctgtaatgccaccaactggattgaatacatgttcaataaggacaatggacaggcaccttttaccatcactcctgtgttttcagattttccagtccatgggatggagcccatgaacaatgccaccaaaggctgtgacgagtctgtggatgaggtcacagcaccatgtagctgccaagactgctctattgtctgtggccccaagccccagcccccacctcctcctgctccctggacgatccttggcttggacgccatgtatgtcatcatgtggatcacctacatggcgtttttgcttgtgttttttggagcattttttgcagtgtggtgctacagaaaacggtattttgtctccgagtacactcccatcgatagcaatatagctttttctgttaatgcaagtgacaaaggagaggcgtcctgctgtgaccctgtcagcgcagcatttgagggctgcttgaggcggctgttcacacgctgggggtctttctgcgtccgaaaccctggctgtgtcattttcttctcgctggtcttcattactgcgtgttcgtcaggcctggtgtttgtccgggtcacaaccaatccagttgacctctggtcagcccccagcagccaggctcgcctggaaaaagagtactttgaccagcactttgggcctttcttccggacggagcagctcatcatccgggcccctctcactgacaaacacatttaccagccatacccttcgggagctgatgtaccctttggacctccgcttgacatacagatactgcaccaggttcttgacttacaaatagccatcgaaaacattactgcctcttatgacaatgagactgtgacacttcaagacatctgcttggcccctctttcaccgtataacacgaactgcaccattttgagtgtgttaaattacttccagaacagccattccgtgctggaccacaagaaaggggacgacttctttgtgtatgccgattaccacacgcactttctgtactgcgtacgggctcctgcctctctgaatgatacaagtttgctccatgacccttgtctgggtacgtttggtggaccagtgttcccgtggcttgtgttgggaggctatgatgatcaaaactacaataacgccactgcccttgtgattaccttccctgtcaataattactataatgatacagagaagctccagagggcccaggcctgggaaaaagagtttattaattttgtgaaaaactacaagaatcccaatctgaccatttccttcactgctgaacgaagtattgaagatgaactaaatcgtgaaagtgacagtgatgtcttcaccgttgtaattagctatgccatcatgtttctatatatttccctagccttggggcacatcaaaagctgtcgcaggcttctggtggattcgaaggtctcactaggcatcgcgggcatcttgatcgtgctgagctcggtggcttgctccttgggtgtcttcagctacattgggttgcccttgaccctcattgtgattgaagtcatcccgttcctggtgctggctgttggagtggacaacatcttcattctggtgcaggcctaccagagagatgaacgtcttcaaggggaaaccctggatcagcagctgggcagggtcctaggagaagtggctcccagtatgttcctgtcatccttttctgagactgtagcatttttcttaggagcattgtccgtgatgccagccgtgcacaccttctctctctttgcgggattggcagtcttcattgactttcttctgcagattacctgtttcgtgagtctcttggggttagacattaaacgtcaagagaaaaatcggctagacatcttttgctgtgtcagaggtgctgaagatggaacaagcgtccaggcctcagagagctgtttgtttcgcttcttcaaaaactcctattctccacttctgctaaaggactggatgagaccaattgtgatagcaatatttgtgggtgttctgtcattcagcatcgcagtcctgaacaaagtagatattggattggatcagtctctttcgatgccagatgactcctacatggtggattatttcaaatccatcagtcagtacctgcatgcgggtccgcctgtgtactttgtcctggaggaagggcacgactacacttcttccaaggggcagaacatggtgtgcggcggcatgggctgcaacaatgattccctggtgcagcagatatttaacgcggcgcagctggacaactatacccgaataggcttcgccccctcgtcctggatcgacgattatttcgactgggtgaagccacagtcgtcttgctgtcgagtggacaatatcactgaccagttctgcaatgcttcagtggttgaccctgcctgcgttcgctgcaggcctctgactccggaaggcaaacagaggcctcaggggggagacttcatgagattcctgcccatgttcctttcggataaccctaaccccaagtgtggcaaagggggacatgctgcctatagttctgcagttaacatcctccttggccatggcaccagggtcggagccacgtacttcatgacctaccacaccgtgctgcagacctctgctgactttattgacgctctgaagaaagcccgacttatagccagtaatgtcaccgaaaccatgggcattaacggcagtgcctaccgagtatttccttacagtgtgttttatgtcttctacgaacagtacctgaccatcattgacgacactatcttcaacctcggtgtgtccctgggcgcgatatttctggtgaccatggtcctcctgggctgtgagctctggtctgcagtcatcatgtgtgccaccatcgccatggtcttggtcaacatgtttggagttatgtggctctggggcatcagtctgaacgctgtatccttggtcaacctggtgatgagctgtggcatctccgtggagttctgcagccacataaccagagcgttcacggtgagcatgaaaggcagccgcgtggagcgcgcggaagaggcacttgcccacatgggcagctccgtgttcagtggaatcacacttacaaaatttggagggattgtggtgttggcttttgccaaatctcaaattttccagatattctacttcaggatgtatttggccatggtcttactgggagccactcacggattaatatttctccctgtcttactcagttacatagggccatcagtaaataaagccaaaagttgtgccactgaagagcgatacaaaggaacagagcgcgaacggcttctaaatttctaggtttaaacaagctttaagatacattgatgagtttggacaaaccacaactagaatgcagtgaaaaaaatgctttatttgtgaaatttgtgatgctattgctttatttgtaaccattataagctgcaataaacaagttctcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 18 - DNA, 4663 bp, Artificial Sequence
  AAV Transfer Casette 5
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgaccggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatgcatgtaagtttagtctttttgtcttttatttcaggtcccggatccggtggtggtgcaaatcaaagaactgctcctcagtggatgttgcctttacttctagcaccggtcgccaccatgaccgctcgcggcctggcccttatgaccgctcgcggccggcctcctcctgctgctactgtgtccagcgcaggtgttttcacagtcctgtgtttggtatggagagtgtggaattgcatatggggacaagaggtacaattgcgaatattctggcccaccaaaaccattgccaaaggatggatatgacttagtgcaggaactctgtccaggattcttctttggcaatgtcagtctctgttgtgatgttcggcagcttcagacactaaaagacaacctgcagctgcctctacagtttctgtccagatgtccatcctgtttttataacctactgaacctgttttgtgagctgacatgtagccctcgacagagtcagtttttgaatgttacagctactgaagattatgttgatcctgttacaaaccagacgaaaacaaatgtgaaagagttacaatactacgtcggacagagttttgccaatgcaatgtacaatgcctgccgggatgtggaggccccctcaagtaatgacaaggccctgggactcctgtgtgggaaggacgctgacgcctgtaatgccaccaactggattgaatacatgttcaataaggacaatggacaggcaccttttaccatcactcctgtgttttcagattttccagtccatgggatggagcccatgaacaatgccaccaaaggctgtgacgagtctgtggatgaggtcacagcaccatgtagctgccaagactgctctattgtctgtggccccaagccccagcccccacctcctcctgctccctggacgatccttggcttggacgccatgtatgtcatcatgtggatcacctacatggcgtttttgcttgtgttttttggagcattttttgcagtgtggtgctacagaaaacggtattttgtctccgagtacactcccatcgatagcaatatagctttttctgttaatgcaagtgacaaaggagaggcgtcctgctgtgaccctgtcagcgcagcatttgagggctgcttgaggcggctgttcacacgctgggggtctttctgcgtccgaaaccctggctgtgtcattttcttctcgctggtcttcattactgcgtgttcgtcaggcctggtgtttgtccgggtcacaaccaatccagttgacctctggtcagcccccagcagccaggctcgcctggaaaaagagtactttgaccagcactttgggcctttcttccggacggagcagctcatcatccgggcccctctcactgacaaacacatttaccagccatacccttcgggagctgatgtaccctttggacctccgcttgacatacagatactgcaccaggttcttgacttacaaatagccatcgaaaacattactgcctcttatgacaatgagactgtgacacttcaagacatctgcttggcccctctttcaccgtataacacgaactgcaccattttgagtgtgttaaattacttccagaacagccattccgtgctggaccacaagaaaggggacgacttctttgtgtatgccgattaccacacgcactttctgtactgcgtacgggctcctgcctctctgaatgatacaagtttgctccatgacccttgtctgggtacgtttggtggaccagtgttcccgtggcttgtgttgggaggctatgatgatcaaaactacaataacgccactgcccttgtgattaccttccctgtcaataattactataatgatacagagaagctccagagggcccaggcctgggaaaaagagtttattaattttgtgaaaaactacaagaatcccaatctgaccatttccttcactgctgaacgaagtattgaagatgaactaaatcgtgaaagtgacagtgatgtcttcaccgttgtaattagctatgccatcatgtttctatatatttccctagccttggggcacatcaaaagctgtcgcaggcttctggtggattcgaaggtctcactaggcatcgcgggcatcttgatcgtgctgagctcggtggcttgctccttgggtgtcttcagctacattgggttgcccttgaccctcattgtgattgaagtcatcccgttcctggtgctggctgttggagtggacaacatcttcattctggtgcaggcctaccagagagatgaacgtcttcaaggggaaaccctggatcagcagctgggcagggtcctaggagaagtggctcccagtatgttcctgtcatccttttctgagactgtagcatttttcttaggagcattgtccgtgatgccagccgtgcacaccttctctctctttgcgggattggcagtcttcattgactttcttctgcagattacctgtttcgtgagtctcttggggttagacattaaacgtcaagagaaaaatcggctagacatcttttgctgtgtcagaggtgctgaagatggaacaagcgtccaggcctcagagagctgtttgtttcgcttcttcaaaaactcctattctccacttctgctaaaggactggatgagaccaattgtgatagcaatatttgtgggtgttctgtcattcagcatcgcagtcctgaacaaagtagatattggattggatcagtctctttcgatgccagatgactcctacatggtggattatttcaaatccatcagtcagtacctgcatgcgggtccgcctgtgtactttgtcctggaggaagggcacgactacacttcttccaaggggcagaacatggtgtgcggcggcatgggctgcaacaatgattccctggtgcagcagatatttaacgcggcgcagctggacaactatacccgaataggcttcgccccctcgtcctggatcgacgattatttcgactgggtgaagccacagtcgtcttgctgtcgagtggacaatatcactgaccagttctgcaatgcttcagtggttgaccctgcctgcgttcgctgcaggcctctgactccggaaggcaaacagaggcctcaggggggagacttcatgagattcctgcccatgttcctttcggataaccctaaccccaagtgtggcaaagggggacatgctgcctatagttctgcagttaacatcctccttggccatggcaccagggtcggagccacgtacttcatgacctaccacaccgtgctgcagacctctgctgactttattgacgctctgaagaaagcccgacttatagccagtaatgtcaccgaaaccatgggcattaacggcagtgcctaccgagtatttccttacagtgtgttttatgtcttctacgaacagtacctgaccatcattgacgacactatcttcaacctcggtgtgtccctgggcgcgatatttctggtgaccatggtcctcctgggctgtgagctctggtctgcagtcatcatgtgtgccaccatcgccatggtcttggtcaacatgtttggagttatgtggctctggggcatcagtctgaacgctgtatccttggtcaacctggtgatgagctgtggcatctccgtggagttctgcagccacataaccagagcgttcacggtgagcatgaaaggcagccgcgtggagcgcgcggaagaggcacttgcccacatgggcagctccgtgttcagtggaatcacacttacaaaatttggagggattgtggtgttggcttttgccaaatctcaaattttccagatattctacttcaggatgtatttggccatggtcttactgggagccactcacggattaatatttctccctgtcttactcagttacatagggccatcagtaaataaagccaaaagttgtgccactgaagagcgatacaaaggaacagagcgcgaacggcttctaaatttctaggtttaaacaagctttaagatacattgatgagtttggacaaaccacaactagaatgcagtgaaaaaaatgctttatttgtgaaatttgtgatgctattgctttatttgtaaccattataagctgcaataaacaagttctcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 19 - DNA, 4691 bp, Artificial Sequence
  AAV Transfer Casette 6
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgactacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaatgcataccggtcgccaccatgaccgctcgcggcctggcccttggcctcctcctgctgctactgtgtccagcgcaggtgttttcacagtcctgtgtttggtatggagagtgtggaattgcatatggggacaagaggtacaattgcgaatattctggcccaccaaaaccattgccaaaggatggatatgacttagtgcaggaactctgtccaggattcttctttggcaatgtcagtctctgttgtgatgttcggcagcttcagacactaaaagacaacctgcagctgcctctacagtttctgtccagatgtccatcctgtttttataacctactgaacctgttttgtgagctgacatgtagccctcgacagagtcagtttttgaatgttacagctactgaagattatgttgatcctgttacaaaccagacgaaaacaaatgtgaaagagttacaatactacgtcggacagagttttgccaatgcaatgtacaatgcctgccgggatgtggaggccccctcaagtaatgacaaggccctgggactcctgtgtgggaaggacgctgacgcctgtaatgccaccaactggattgaatacatgttcaataaggacaatggacaggcaccttttaccatcactcctgtgttttcagattttccagtccatgggatggagcccatgaacaatgccaccaaaggctgtgacgagtctgtggatgaggtcacagcaccatgtagctgccaagactgctctattgtctgtggccccaagccccagcccccacctcctcctgctccctggacgatccttggcttggacgccatgtatgtcatcatgtggatcacctacatggcgtttttgcttgtgttttttggagcattttttgcagtgtggtgctacagaaaacggtattttgtctccgagtacactcccatcgatagcaatatagctttttctgttaatgcaagtgacaaaggagaggcgtcctgctgtgaccctgtcagcgcagcatttgagggctgcttgaggcggctgttcacacgctgggggtctttctgcgtccgaaaccctggctgtgtcattttcttctcgctggtcttcattactgcgtgttcgtcaggcctggtgtttgtccgggtcacaaccaatccagttgacctctggtcagcccccagcagccaggctcgcctggaaaaagagtactttgaccagcactttgggcctttcttccggacggagcagctcatcatccgggcccctctcactgacaaacacatttaccagccatacccttcgggagctgatgtaccctttggacctccgcttgacatacagatactgcaccaggttcttgacttacaaatagccatcgaaaacattactgcctcttatgacaatgagactgtgacacttcaagacatctgcttggcccctctttcaccgtataacacgaactgcaccattttgagtgtgttaaattacttccagaacagccattccgtgctggaccacaagaaaggggacgacttctttgtgtatgccgattaccacacgcactttctgtactgcgtacgggctcctgcctctctgaatgatacaagtttgctccatgacccttgtctgggtacgtttggtggaccagtgttcccgtggcttgtgttgggaggctatgatgatcaaaactacaataacgccactgcccttgtgattaccttccctgtcaataattactataatgatacagagaagctccagagggcccaggcctgggaaaaagagtttattaattttgtgaaaaactacaagaatcccaatctgaccatttccttcactgctgaacgaagtattgaagatgaactaaatcgtgaaagtgacagtgatgtcttcaccgttgtaattagctatgccatcatgtttctatatatttccctagccttggggcacatcaaaagctgtcgcaggcttctggtggattcgaaggtctcactaggcatcgcgggcatcttgatcgtgctgagctcggtggcttgctccttgggtgtcttcagctacattgggttgcccttgaccctcattgtgattgaagtcatcccgttcctggtgctggctgttggagtggacaacatcttcattctggtgcaggcctaccagagagatgaacgtcttcaaggggaaaccctggatcagcagctgggcagggtcctaggagaagtggctcccagtatgttcctgtcatccttttctgagactgtagcatttttcttaggagcattgtccgtgatgccagccgtgcacaccttctctctctttgcgggattggcagtcttcattgactttcttctgcagattacctgtttcgtgagtctcttggggttagacattaaacgtcaagagaaaaatcggctagacatcttttgctgtgtcagaggtgctgaagatggaacaagcgtccaggcctcagagagctgtttgtttcgcttcttcaaaaactcctattctccacttctgctaaaggactggatgagaccaattgtgatagcaatatttgtgggtgttctgtcattcagcatcgcagtcctgaacaaagtagatattggattggatcagtctctttcgatgccagatgactcctacatggtggattatttcaaatccatcagtcagtacctgcatgcgggtccgcctgtgtactttgtcctggaggaagggcacgactacacttcttccaaggggcagaacatggtgtgcggcggcatgggctgcaacaatgattccctggtgcagcagatatttaacgcggcgcagctggacaactatacccgaataggcttcgccccctcgtcctggatcgacgattatttcgactgggtgaagccacagtcgtcttgctgtcgagtggacaatatcactgaccagttctgcaatgcttcagtggttgaccctgcctgcgttcgctgcaggcctctgactccggaaggcaaacagaggcctcaggggggagacttcatgagattcctgcccatgttcctttcggataaccctaaccccaagtgtggcaaagggggacatgctgcctatagttctgcagttaacatcctccttggccatggcaccagggtcggagccacgtacttcatgacctaccacaccgtgctgcagacctctgctgactttattgacgctctgaagaaagcccgacttatagccagtaatgtcaccgaaaccatgggcattaacggcagtgcctaccgagtatttccttacagtgtgttttatgtcttctacgaacagtacctgaccatcattgacgacactatcttcaacctcggtgtgtccctgggcgcgatatttctggtgaccatggtcctcctgggctgtgagctctggtctgcagtcatcatgtgtgccaccatcgccatggtcttggtcaacatgtttggagttatgtggctctggggcatcagtctgaacgctgtatccttggtcaacctggtgatgagctgtggcatctccgtggagttctgcagccacataaccagagcgttcacggtgagcatgaaaggcagccgcgtggagcgcgcggaagaggcacttgcccacatgggcagctccgtgttcagtggaatcacacttacaaaatttggagggattgtggtgttggcttttgccaaatctcaaattttccagatattctacttcaggatgtatttggccatggtcttactgggagccactcacggattaatatttctccctgtcttactcagttacatagggccatcagtaaataaagccaaaagttgtgccactgaagagcgatacaaaggaacagagcgcgaacggcttctaaatttctaggtttaaacaagcttaataaaggaaatttattttcattgcaatagtgtgttggaattttttgtgtctctcactcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 20 - protein, 1278 aa, Artificial Sequence
  Variant NPC1 protein
MTARGLALGLLLLLLCPAQVFSQSCVWYGECGIAYGDKRYNCEYSGPPKPLPKDGYDLVQELCPGFFFGNVSLCCDVRQLQTLKDNLQLPLQFLSRCPSCFYNLLNLFCELTCSPRQSQFLNVTATEDYVDPVTNQTKTNVKELQYYVGQSFANAMYNACRDVEAPSSNDKALGLLCGKDADACNATNWIEYMFNKDNGQAPFTITPVFSDFPVHGMEPMNNATKGCDESVDEVTAPCSCQDCSIVCGPKPQPPPPPAPWTILGLDAMYVIMWITYMAFLLVFFGAFFAVWCYRKRYFVSEYTPIDSNIAFSVNASDKGEASCCDPVSAAFEGCLRRLFTRWGSFCVRNPGCVIFFSLVFITACSSGLVFVRVTTNPVDLWSAPSSQARLEKEYFDQHFGPFFRTEQLIIRAPLTDKHIYQPYPSGADVPFGPPLDIQILHQVLDLQIAIENITASYDNETVTLQDICLAPLSPYNTNCTILSVLNYFQNSHSVLDHKKGDDFFVYADYHTHFLYCVRAPASLNDTSLLHDPCLGTFGGPVFPWLVLGGYDDQNYNNATALVITFPVNNYYNDTEKLQRAQAWEKEFINFVKNYKNPNLTISFTAERSIEDELNRESDSDVFTVVISYAIMFLYISLALGHMKSCRRLLVDSKVSLGIAGILIVLSSVACSLGVFSYIGLPLTLIVIEVIPFLVLAVGVDNIFILVQAYQRDERLQGETLDQQLGRVLGEVAPSMFLSSFSETVAFFLGALSVMPAVHTFSLFAGLAVFIDFLLQITCFVSLLGLDIKRQEKNRLDIFCCVRGAEDGTSVQASESCLFRFFKNSYSPLLLKDWMRPIVIAIFVGVLSFSIAVLNKVDIGLDQSLSMPDDSYMVDYFKSISQYLHAGPPVYFVLEEGHDYTSSKGQNMVCGGMGCNNDSLVQQIFNAAQLDNYTRIGFAPSSWIDDYFDWVKPQSSCCRVDNITDQFCNASVVDPACVRCRPLTPEGKQRPQGGDFMRFLPMFLSDNPNPKCGKGGHAAYSSAVNILLGHGTRVGATYFMTYHTVLQTSADFIDALKKARLIASNVTETMGINGSAYRVFPYSVFYVFYEQYLTIIDDTIFNLGVSLGAIFLVTMVLLGCELWSAVIMCATIAMVLVNMFGVMWLWGISLNAVSLVNLVMSCGISVEFCSHITRAFTVSMKGSRVERAEEALAHMGSSVFSGITLTKFGGIVVLAFAKSQIFQIFYFRMYLAMVLLGATHGLIFLPVLLSYIGPSVNKAKSCATEERYKGTERERLLNF

(19) United States
(12) Patent Application Publication
(10) Pub. No.: US 2024/0226206 A9
(43) Pub. Date: Jul. 11, 2024

(54) AAV TRANSFER CASSETTE

(71) Applicant: StrideBio, Inc., Research Triangle, NC (US)

(72) Inventor: David DISMUKE, Cary, NC (US)

(21) Appl. No.: 17/769,615

(22) PCT Filed: Oct. 16, 2020
(86) PCT No.: PCT/US2020/056015

§ 371 Date: Apr. 15, 2022

Related U.S. Application Data
(60) Provisional application No. 62/916,749, filed on Oct. 17, 2019.
(60) Provisional application No. 62/923,253, filed on Oct. 18, 2019.

(51) Int. Cl. A61K 48/00; A61P 25/28; C07K 14/705; C12N 15/86

(52) U.S. Cl. CPC A61K 48/005; A61P 25/28; C07K 14/705; C12N 15/86; C12N 2750/14143

(57) ABSTRACT

Described herein are AAV transfer cassettes and plasmids used in the production of recombinant adeno-associated viral (rAAV) vectors. The disclosed cassettes and plasmids comprise one or more transgenes having therapeutic efficacy in the amelioration, treatment and/or prevention of one or more diseases or disorders, such as Niemann-Pick Disease, type C1 (NPC1).

### CROSS-REFERENCE TO RELATED APPLICATIONS

This application claims priority to U.S. Provisional Application No. 62/923,253, filed on Oct. 18, 2019, and U.S. Provisional Application No. 62/916,749, filed on Oct. 17, 2019, each of which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is STRD_015_02WO_SeqList_ST25.txt. The file is ˜67.6 kb, was created on Oct. 14, 2020, and is being submitted electronically.

### FIELD

The instant disclosure relates to the fields of molecular biology and gene therapy. More specifically, disclosure relates to compositions and methods for producing recombinant viral vectors.

### BACKGROUND

Niemann-Pick Disease, type C1 (NPC1) is a neurodegenerative disorder characterized by cholesterol accumulation in endolysosomal compartments. It is caused by mutations in the gene encoding NPC1, an endolysosomal protein mediating intracellular cholesterol trafficking.

NPC1 can present in infants, children, or adults. Neonates can present with ascites and severe liver disease from infiltration of the liver and/or respiratory failure from infiltration of the lungs. Other infants, without liver or pulmonary disease, have hypotonia and developmental delay. The classic presentation occurs in mid-to-late childhood with the insidious onset of ataxia, vertical supranuclear gaze palsy (VSGP), and dementia. Dystonia and seizures are common. Dysarthria and dysphagia eventually become disabling, making oral feeding impossible; death usually occurs in the late second or third decade from aspiration pneumonia. Adults are more likely to present with dementia or psychiatric symptoms.

2-hydroxypropyl-β-cyclodextrin (HPBCD) has been shown to reduce the cholesterol and lipid accumulation and prolongs survival in NPC1 animal models. However, there are no therapies for NPC1 approved by the Food and Drug Administration (FDA). Accordingly, there is an urgent need for compositions and methods for treating, curing, and/or preventing NPC1.

### SUMMARY

Described herein are AAV transfer cassettes, nucleic acids and plasmids used in the production of recombinant adeno-associated viral (rAAV) vectors for the delivery of nucleic acids (e.g., nucleic acids comprising transgenes). The disclosed cassettes, nucleic acids and plasmids comprise sequences that may be used to express one or more transgenes having therapeutic efficacy in the amelioration, treatment and/or prevention of one or more diseases or disorders, such as NPC1.

In some embodiments, the disclosure provides an adeno-associated virus (AAV) transfer cassette comprising a 5′ inverted terminal repeat (ITR), a promoter, a transgene, a polyadenylation signal, and a 3′ ITR, wherein the transfer cassette comprises an intronic sequence. In some embodiments, the intronic sequence is located between the promoter and the transgene. In some embodiments, the transgene encodes the NPC1 protein. In some embodiments, the AAV transfer cassette comprises the sequence of any one of SEQ ID NO: 14-19.

In some embodiments, the disclosure provides a recombinant AAV vector comprising a protein capsid and a nucleic acid encapsidated by the protein capsid, wherein the nucleic acid comprises an transfer cassette comprising, from 5′ to 3′, a 5′ inverted terminal repeat (ITR); a promoter; a transgene; a polyadenylation signal; and a 3′ ITR. In some embodiments, the transfer cassette comprises an intronic sequence, such as an intronic sequence located between the promoter and the transgene. In some embodiments, the transgene encodes the NPC1 protein. In some embodiments, the AAV transfer cassette comprises the sequence of any one of SEQ ID NO: 14-19.

In some embodiments, at least one of the 5′ ITR and the 3′ ITR is about 110 to about 160 nucleotides in length. In some embodiments, the 5′ ITR is the same length as the 3′ ITR. In some embodiments, the 5′ ITR and the 3′ ITR have different lengths. At least one of the 5′ ITR and the 3′ ITR may be isolated or derived from, for example, the genome of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV. In some embodiments, the 5′ ITR comprises the sequence of SEQ ID NO: 3, or a sequence at least 95%, at least 96%, at least 97%, at least 98%, or at least 99% identical thereto. In some embodiments, the 3′ ITR comprises the sequence of SEQ ID NO: 4, or a sequence that is at least 95%, at least 96%, at least 97%, at least 98%, or at least 99% identical thereto.

In some embodiments, the promoter is a constitutive promoter. In some embodiments, the promoter is an inducible promoter. In some embodiments, the promoter is a tissue-specific promoter. In some embodiments, the promoter may be selected from the group consisting of the CBA promoter, the GUSB240 promoter, the GUSB379 promoter, the HSVTK promoter, the CMV promoter, the SV40 early promoter, the SV40 late promoter, the metallothionein promoter, the murine mammary tumor virus (MMTV) promoter, the Rous sarcoma virus (RSV) promoter, the polyhedrin promoter, the chicken β-actin (CBA) promoter, the EF-1 alpha promoter, the EF-1 short promoter, the dihydrofolate reductase (DHFR) promoter, and the phosphoglycerol kinase (PGK) promoter. In some embodiments, the promoter is selected from the group consisting of the CBA promoter, the GUSB240 promoter, the GUSB379 promoter, and the HSVTK promoter. In some embodiments, the promoter comprises a sequence at least 95%, at least 96%, at least 97%, at least 98% at least 99%, or 100% identical to any one of SEQ ID NO: 5-8.

In some embodiments, the intronic sequence is a chimeric sequence or a hybrid sequence. In some embodiments, the intronic sequence comprises sequences isolated or derived from SV40. In some embodiments, the intronic sequence comprises the sequence of any one of SEQ ID NO: 10-11.

The NPC1 protein may be, for example, the human NPC1 protein. In some embodiments, the NPC1 protein has a sequence that is at least 90%, at least 95%, at least 95%, at least 97%, at least 98%, or at least 99% identical to the sequence of the human NPC1 protein. In some embodiments, the NPC1 protein comprises the sequence of SEQ ID NO: 1, or a sequence at least 95%, at least 96%, at least 97%, at least 98%, or at least 99% identical thereto. In some embodiments, the NPC1 protein comprises the sequence of SEQ ID NO: 20, or a sequence at least 95%, at least 96%, at least 97%, at least 98%, or at least 99% identical thereto. In some embodiments, the NPC1 protein has a sequence as shown in UniProt Accession No. O15118, incorporated herein by reference in its entirety.

In some embodiments, the transgene comprises a sequence of SEQ ID NO: 2, or a sequence at least 95%, at least 96%, at least 97%, at least 98%, or at least 99% identical thereto.

In some embodiments, the polyadenylation signal is selected from simian virus 40 (SV40), rabbit beta globin (rBG), α-globin, β-globin, human collagen, human growth hormone (hGH), polyoma virus, human growth hormone (hGH) and bovine growth hormone (bGH). In some embodiments, the polyadenylation signal is the SV40 polyadenylation signal. In some embodiments, the polyadenylation signal is the rBG polyadenylation signal. In some embodiments, the polyadenylation signal comprises the sequence of SEQ ID NO: 12 or SEQ ID NO: 13. In some embodiments, the polyadenylation signal comprises a sequence at least 95%, at least 96%, at least 97%, at least 98%, or at least 99% identical to SEQ ID NO: 12 or 13.

In some embodiments, the AAV transfer cassette further comprises an enhancer. The enhancer may be, for example, the CMV enhancer. In some embodiments, the enhancer comprises the sequence of SEQ ID NO: 9, or a sequence at least 95%, at least 96%, at least 97%, at least 98%, or at least 99% identical thereto.

Also provided herein is a nucleic acid comprising an AAV transfer cassette of the disclosure. Also provided herein is a plasmid or bacmid comprising an AAV transfer cassette of the disclosure.

Also provided herein is a recombinant AAV vector comprising a protein capsid and a nucleic acid encapsidated by the protein capsid, wherein the nucleic acid comprises an AAV transfer cassette of the disclosure.

Also provided is a cell comprising the AAV transfer cassette of the disclosure.

Also provided is a method of producing a recombinant AAV vector, the method comprising contacting an AAV producer cell with an AAV transfer cassette or plasmid/bacmid of the disclosure. Also provided is a recombinant AAV vector produced by this method. The recombinant AAV vector may be of a serotype selected from AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV and Bovine AAV. The recombinant AAV vector may comprise a protein capsid comprising a capsid protein subunit, wherein the capsid protein subunit comprises one or more mutations compared to a capsid protein subunit of a wildtype AAV.

Also provided are compositions comprising an AAV transfer cassette, a nucleic acid (e.g., a plasmid or a bacmid), a cell, or a recombinant AAV vector of the disclosure.

Also provided is a method for treating a subject in need thereof comprising administering to the subject a therapeutically effective amount of an AAV transfer cassette, a nucleic acid (e.g., a plasmid), a cell, or a recombinant AAV vector of the disclosure. In some embodiments, the subject is a human subject. In some embodiments, the subject has NPC1.

These and other embodiments are addressed in more detail in the detailed description set forth below.

### BRIEF DESCRIPTION OF THE DRAWINGS

FIG. 1A is a graph that shows lysosomal phenotype, as determined by measuring LysoTracker® accumulation, in wildtype U2OS cells, NPC1-deficient (NPC1^{-/-}) U2OS cells, and NPC1^{-/-} cells transduced with AAV2-hNPC at a Multiplicity of Infection (MOI) of either 5×10³ or 10×10³. Statistical significance determined using one-way ANOVA. Error bars represent standard error of the mean (SEM).

FIG. 1B is a graph that shows cholesterol accumulation, as determined using filipin staining, in wildtype U20S cells, NPC1-deficient (NPC1^{-/-}) U20S cells, and NPC1^{-/-} cells transduced with AAV2-hNPC at a Multiplicity of Infection (MOI) of either 5×10³ or 10×10³. Statistical significance determined using one-way ANOVA. Error bars represent SEM.

FIG. 2 is a Kaplan-Meier survival curve, showing survival of NPC1^{-/-} mice after retro-orbital injection with saline or with AAV9-hNPC1. All AAV9-hNPC1-injected animals survived through the duration of the experiment, and were sacrificed around 100 days of age for histological analysis.

FIG. 3 shows behavioral phenotype score at about 10 weeks (70 days) of age in wildtype mice, saline-treated NPC1^{-/-} mice, or NPC1^{-/-} mice injected with AAV9-hNPC1. Statistical significance was determined using an unpaired T-test, and error bars represent SEM.

FIG. 4 shows number of slips in a balance beam walking test at about 8 weeks (56 days) of age in wildtype mice, saline-treated NPC1^{-/-} mice, or NPC1^{-/-} mice treated with AAV9-hNPC1. Error bars represent standard deviation.

### DETAILED DESCRIPTION

Provided herein are gene therapy compositions and methods for treating, preventing, and/or curing NPC1. More specifically, the disclosure provides Adeno-associated virus (AAV) vectors for the delivery of nucleic acids, e.g. transgenes, and nucleic acids (including AAV transfer cassettes) for treating, preventing, and/or curing NPC1.

AAVs are useful as gene delivery agents, and are powerful tools for human gene therapy. Using AAVs, high-frequency DNA delivery and stable expression may be achieved in a variety of cells, both in vivo and in vitro. Unlike some other viral vector systems, AAV does not require active cell division for stable integration in target cells.

All papers, publications and patents cited in this specification are herein incorporated by reference as if each individual paper, publication or patent were specifically and individually indicated to be incorporated by reference and are incorporated herein by reference to disclose and describe the methods and/or materials in connection with which the publications are cited.

Unless the context indicates otherwise, it is specifically intended that the various features described herein can be used in any combination.

Unless otherwise defined, all technical and scientific terms used herein have the same meaning as commonly understood by one of ordinary skill in the art to which this disclosure belongs. The terminology used in the detailed description herein is for the purpose of describing particular embodiments only and is not intended to be limiting.

### Definitions

The following terms are used in the description herein and the appended claims:
The singular forms “a,” “an” and “the” are intended to include the plural forms as well, unless the context clearly indicates otherwise.

Furthermore, the term “about” as used herein when referring to a measurable value such as an amount of the length of a polynucleotide or polypeptide sequence, dose, time, temperature, and the like, is meant to encompass variations of ±20%, 10%, 5%, 1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

A “nucleic acid” or “polynucleotide” is a sequence of nucleotide bases, for example RNA, DNA or DNA-RNA hybrid sequences (including both naturally occurring and non-naturally occurring nucleotides). In some embodiments, the nucleic acids of the disclosure are either single or double stranded DNA sequences. A nucleic acid may be 1-1,000, 1,000-10,000, 10,000-100,000, 100,000-1 million or greater than 1 million nucleotides in length. A nucleic acid will generally contain phosphodiester bonds, although in some cases nucleic acid analogs are included that may have alternate backbones, comprising, for example, phosphoramide, phosphorothioate, phosphorodithioate, O-methylphophoroamidite linkages, and peptide nucleic acid backbones and linkages. Other analog nucleic acids include those with positive backbones, non-ionic backbones, and non-ribose backbones. Nucleic acids containing one or more carbocyclic sugars are also included within the definition of nucleic acids. These modifications of the ribose-phosphate backbone may facilitate the addition of labels, or to increase the stability and half-life of such molecules in physiological environments. Nucleic acids of the disclosure may be linear, or may be circular (e.g., a plasmid).

An “AAV transfer cassette” is a nucleic acid that may be used in the generation of an AAV vector.

As used herein, the terms “virus vector,” or “viral vector” refer to a virus particle that functions as a nucleic acid delivery vehicle, and which comprises a nucleic acid (e.g., a nucleic acid comprising a transgene) packaged within a virion or virus-like particle. Exemplary virus vectors of the disclosure include adenovirus vectors, adeno-associated virus vectors (AAVs), lentivirus vectors, and retrovirus vectors.

An “adeno-associated virus vector” or “AAV vector” typically comprises a protein capsid, and a nucleic acid (e.g., a nucleic acid comprising a transgene) encapsidated by the protein capsid. The “protein capsid” is a near-spherical protein shell that comprises individual “capsid protein subunits” (e.g., about 60 capsid protein subunits) associated and arranged with T=1 icosahedral symmetry. The protein capsids of the AAV vectors described herein comprise a plurality of capsid protein subunits. When an AAV vector is described herein as comprising an AAV capsid protein subunit, it will be understood that the AAV vector comprises a protein capsid, wherein the protein capsid comprises one or more AAV capsid protein subunits. As used herein, the term “capsid protein” is sometimes used to refer to a capsid protein subunit. The term “viral-like particle” or “virus-like particle” refers to a protein capsid that does not comprise any vector genome or nucleic acid comprising a transfer cassette or transgene.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, AAV type rh74, AAV type hu.68, avian AAV, bovine AAV, canine AAV, equine AAV, ovine AAV, snake AAV, bearded dragon AAV, AAV2i8, AAV2g9, AAV-LKO3, AAV7m8, AAV Anc80, AAV PHP.B, and any other AAV now known or later discovered. See, e.g., Table 1.

TABLE 1

| | GenBank | | GenBank | | GenBank |
|---|---|---|---|---|---|
| | Accession | | Accession | | Accession |
| | Number | | Number | | Number |
| Complete | | Clade C | | Rh57 | AY530569 |
| Genomes | | | | | |
| Adeno-associated | NC_002077, | Hu9 | AY530629 | Rh50 | AY530563 |
| virus 1 | AF063497 | | | | |
| Adeno-associated | NC_001401 | Hu10 | AY530576 | Rh49 | AY530562 |
| virus 2 | | | | | |
| Adeno-associated | NC_001729 | Hu11 | AY530577 | Hu39 | AY530601 |
| virus 3 | | | | | |
| Adeno-associated | NC_001863 | Hu53 | AY530615 | Rh58 | AY530570 |
| virus 3B | | | | | |
| Adeno-associated | NC_001829 | Hu55 | AY530617 | Rh61 | AY530572 |
| virus 4 | | | | | |
| Adeno-associated | Y18065, | Hu54 | AY530616 | Rh52 | AY530565 |
| virus 5 | AF085716 | | | | |
| Adeno-associated | NC_001862, | Hu7 | AY530628 | Rh53 | AY530566 |
| virus 6 | AAB95450.1 | | | | |
| Avian AAV ATCC | AY186198, | Hu18 | AY530583 | Rh51 | AY530564 |
| VR-865 | AY629583, | | | | |
| | NC_004828 | | | | |
| Avian AAV strain | NC_006263, | Hu15 | AY530580 | Rh64 | AY530574 |
| DA-1 | AY629583 | | | | |
| Bovine AAV | NC_005889, | Hu16 | AY530581 | Rh43 | AY530560 |
| | AY388617, | | | | |
| | AAR26465 | | | | |
| AAV11 | AAT46339, | Hu25 | AY530591 | AAV8 | AF513852 |
| | AY631966 | | | | |
| AAV12 | ABI16639, | Hu60 | AY530622 | Rh8 | AY242997 |
| | DQ813647 | | | | |
| Clade A | | Ch5 | AY243021 | Rh1 | AY530556 |
| AAV1 | NC_002077, | Hu3 | AY530595 | Clade F | |
| | AF063497 | | | | |
| AAV6 | NC_001862 | Hu1 | AY530575 | Hu14 | AY530579 |
| | | | | (AAV9) | |
| Hu.48 | AY530611 | Hu4 | AY530602 | Hu31 | AY530596 |
| Hu 43 | AY530606 | Hu2 | AY530585 | Hu32 | AY530597 |
| Hu 44 | AY530607 | Hu61 | AY530623 | HSC1 | MI332400.1 |
| Hu 46 | AY530609 | Clade D | | HSC2 | MI332401.1 |
| Clade B | | Rh62 | AY530573 | HSC3 | MI332402.1 |
| Hu. 19 | AY530584 | Rh48 | AY530561 | HSC4 | MI332403.1 |
| Hu. 20 | AY530586 | Rh54 | AY530567 | HSC5 | MI332405.1 |
| Hu 23 | AY530589 | Rh55 | AY530568 | HSC6 | MI332404.1 |
| Hu22 | AY530588 | Cy2 | AY243020 | HSC7 | MI332407.1 |
| Hu24 | AY530590 | AAV7 | AF513851 | HSC8 | MI332408.1 |
| Hu21 | AY530587 | Rh35 | AY243000 | HSC9 | MI332409.1 |
| Hu27 | AY530592 | Rh37 | AY242998 | HSC11 | MI332406.1 |
| Hu28 | AY530593 | Rh36 | AY242999 | HSC12 | MI332410.1 |
| Hu 29 | AY530594 | Cy6 | AY243016 | HSC13 | MI332411.1 |
| Hu63 | AY530624 | Cy4 | AY243018 | HSC14 | MI332412.1 |
| Hu64 | AY530625 | Cy3 | AY243019 | HSC15 | MI332413.1 |
| Hu13 | AY530578 | Cy5 | AY243017 | HSC16 | MI332414.1 |
| Hu56 | AY530618 | Rh13 | AY243013 | HSC17 | MI332415.1 |
| Hu57 | AY530619 | Clade E | | Hu68 | |
| Hu49 | AY530612 | Rh38 | AY530558 | Clonal | |
| | | | | Isolate | |
| Hu58 | AY530620 | Hu66 | AY530626 | AAV5 | Y18065, |
| | | | | | AF085716 |
| Hu34 | AY530598 | Hu42 | AY530605 | AAV 3 | NC_001729 |
| Hu35 | AY530599 | Hu67 | AY530627 | AAV 3B | NC_001863 |
| AAV2 | NC_001401 | Hu40 | AY530603 | AAV4 | NC_001829 |
| Hu45 | AY530608 | Hu41 | AY530604 | Rh34 | AY243001 |
| Hu47 | AY530610 | Hu37 | AY530600 | Rh33 | AY243002 |
| Hu51 | AY530613 | Rh40 | AY530559 | Rh32 | AY243003 |
| Hu52 | AY530614 | Rh2 | AY243007 | Others | |
| Hu T41 | AY695378 | Bb1 | AY243023 | Rh74 | |
| Hu S17 | AY695376 | Bb2 | AY243022 | Bearded | |
| | | | | Dragon | |
| | | | | AAV | |
| Hu T88 | AY695375 | Rh10 | AY243015 | Snake | NC_006148.1 |
| | | | | AAV | |
| Hu T71 | AY695374 | Hu17 | AY530582 | | |
| Hu T70 | AY695373 | Hu6 | AY530621 | | |
| Hu T40 | AY695372 | Rh25 | AY530557 | | |
| Hu T32 | AY695371 | Pi2 | AY530554 | | |
| Hu T17 | AY695370 | Pi1 | AY530553 | | |
| Hu LG15 | AY695377 | Pi3 | AY530555 | | |

Recombinant AAV (rAAV) vectors can be produced in culture using viral production cell lines. The terms “viral production cell”, “viral production cell line,” or “viral producer cell” refer to cells used to produce viral vectors. HEK293 and 239T cells are common viral production cell lines. Table 2, below, lists exemplary viral production cell lines for various viral vectors. Production of rAAVs typically requires the presence of three elements in the cells: 1) a transgene flanked by AAV inverted terminal repeat (ITR) sequences, 2) AAV rep and cap genes, and 3) helper virus protein sequences. These three elements may be provided on one or more plasmids, and transfected or transduced into the cells.

TABLE 2
Exemplary viral production cell lines

| | Exemplary Viral |
|---|---|
| Virus Vector | Production Cell Line(s) |
| Adenovirus | HEK293, 911, pTG6559, |
| | PER.C6, GH329, N52.E6, |
| | HeLa-E1, UR, VLI-293 |
| Adeno-Associated Virus (AAV) | HEK293, Sf9 |
| Retrovirus | HEK293 |
| Lentivirus | 293T |

“HEK293” refers to a cell line originally derived from human embryonic kidney cells grown in tissue culture. The HEK293 cell line grows readily in culture, and is commonly used for viral production. As used herein, “HEK293” may also refer to one or more variant HEK293 cell lines, i.e., cell lines derived from the original HEK293 cell line that additionally comprise one or more genetic alterations. Many variant HEK293 lines have been developed and optimized for one or more particular applications. For example, the 293T cell line contains the SV40 large T-antigen that allows for episomal replication of transfected plasmids containing the SV40 origin of replication, leading to increased expression of desired gene products.

“Sf9” refers to an insect cell line that is a clonal isolate derived from the parental Spodoptera frugiperda cell line IPLB-Sf-21-AE. Sf9 cells can be grown in the absence of serum and can be cultured attached or in suspension.

A “transfection reagent” means a composition that enhances the transfer of nucleic acid into cells. Some transfection reagents commonly used in the art include one or more lipids that bind to nucleic acids and to the cell surface (e.g., Lipofectamine™) As used herein, the term “multiplicity of infection” or “MOI” refers to number of virions contacted with a cell. For example, cultured cells may be contacted with AAVs at an MOI in the range of 1×10² to 1×10⁵ virions per cell.

As used herein, an “effective amount” is the amount of an AAV vector, nucleic acid, or other agent provided herein that is effective to treat or prevent a disease or disorder in a subject or to ameliorate a sign or symptom thereof. The “effective amount” may vary depending, for example, on the disease and/or symptoms of the disease, severity of the disease and/or symptoms of the disease or disorder, the age, weight, and/or health of the patient to be treated, and the judgment of the prescribing physician. An appropriate amount in any given instance may be ascertained by those skilled in the art or may be capable of determination by routine experimentation.

Methods of determining sequence similarity or identity between two or more amino acid sequences are known in the art. Sequence similarity or identity may be determined for the entire length of a nucleic acid or an indicated portion of a nucleic acid. Sequence similarity or identity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12, 387-395 (1984), or by inspection.

Another suitable algorithm is the BLAST algorithm, described in Altschul et al., J Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); h t t p: / / blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity.

Further, an additional useful algorithm is gapped BLAST as reported by Altschul et al, (1997) Nucleic Acids Res. 25, 3389-3402.

For purposes of the instant disclosure, unless otherwise indicated, percent identity is calculated using the Basic Local Alignment Search Tool (BLAST) available online at blast.ncbi.nlm.nih.gov/Blast.cgi. The skilled artisan will understand that other algorithms may be substituted as appropriate.

Inverted Terminal Repeat Inverted Terminal Repeat or ITR sequences are sequences that mediate AAV proviral integration and for packaging of AAV DNA into virions. ITRs are involved in a variety of activities in the AAV life cycle. For example, the ITR sequences, which can form a hairpin structure, play roles in excision from the plasmid after transfection, replication of the vector genome, and integration and rescue from a host cell genome.

The AAV transfer cassettes of the disclosure may comprise a 5′ ITR and a 3′ ITR. The ITR sequences may be about 110 to about 160 nucleotides in length, for example 110,111,112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159 or 160 nucleotides in length. In some embodiments, the ITR sequences may be about 141 nucleotides in length. In some embodiments, the 5′ ITR is the same length as the 3′ ITR. In some embodiments, the 5′ ITR and the 3′ ITR have different lengths. In some embodiments, the 5′ ITR is longer than the 3′ ITR, and in other embodiments, the 3′ ITR is longer than the 5′ ITR.

The ITRs may be isolated or derived from the genome of any AAV, for example the AAVs listed in Table 1. In some embodiments, at least one of the 5′ ITR and the 3′ ITR is isolated or derived from the genome of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV. In some embodiments, at least one of the 5′ ITR and the 3′ITR may be a wildtype or mutated ITR isolated derived from a member of another parvovirus species besides AAV. For example, in some embodiments, an ITR may be a wildtype or mutant ITR isolated or derived from bocavirus or parvovirus B19.

In some embodiments, the ITR comprises a modification to promote production of a scAAV. In some embodiments, the modification to promote production of a scAAV is deletion of the terminal resolution sequence (TRS) from the ITR. In some embodiments, the 5′ ITR is a wildtype ITR, and the 3′ ITR is a mutated ITR lacking the terminal resolution sequence. In some embodiments, the 3′ ITR is a wildtype ITR, and the 5′ ITR is a mutated ITR lacking the terminal resolution sequence. In some embodiments, the terminal resolution sequence is absent from both the 5′ ITR and the 3′ITR. In other embodiments, the modification to promote production of a scAAV is replacement of an ITR with a different hairpin-forming sequence, such as a shRNA-forming sequence.

In some embodiments, the 5′ ITR may comprise the sequence of SEQ ID NO: 3, or a sequence at least 95% identical thereto. In some embodiments, the 3′ ITR may comprise the sequence of SEQ ID NO: 4, or a sequence at least 95% identical thereto. In some embodiments, the 5′ ITR comprises the sequence of SEQ ID NO: 3, and the 3′ ITR comprises the sequence of SEQ ID NO: 4.

In some embodiments, the AAV transfer cassettes comprise one or more “surrogate” ITRs, i.e., non-ITR sequences that serve the same function as ITRs. See, e.g., Xie, J. et al., Mol. Ther., 25(6): 1363-1374 (2017). In some embodiments, an ITR in an AAV transfer cassette is replaced by a surrogate ITR. In some embodiments, the surrogate ITR comprises a hairpin-forming sequence. In some embodiments, the surrogate ITR is a short hairpin (sh)RNA-forming sequence.

### Promoters, Enhancers, Repressors and Other Regulatory Sequences

Gene expression may be controlled by nucleotide sequences called promoters and enhancers that flank the coding region for a given protein.

As used herein, the term “promoter” refers to one or more nucleic acid control sequences that direct transcription of an operably linked nucleic acid. Promoters may include nucleic acid sequences near the start site of transcription, such as a TATA element. Promoters may also include cis-acting polynucleotide sequences that can be bound by transcription factors.

A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

Gene expression may also be controlled by one or more distal “enhancer” or “repressor” elements, which can be located as much as several thousand base pairs from the start site of transcription. Enhancer or repressor elements regulate transcription in an analogous manner to cis-acting elements near the start site of transcription, with the exception that enhancer elements can act from a distance from the start site of transcription.

In some embodiments, the AAV transfer cassettes described herein comprise a promoter. They promoter may be, for example, a constitutive promoter or an inducible promoter. In some embodiments, the promoter is a tissue-specific promoter.

Exemplary promoters that may be used in the AAV transfer cassettes described herein include the CMV promoter, the SV40 early promoter, the SV40 late promoter, the metallothionein promoter, the murine mammary tumor virus (MMTV) promoter, the Rous sarcoma virus (RSV) promoter, the polyhedrin promoter, the chicken β-actin (CBA) promoter, the dihydrofolate reductase (DHFR) promoter, and the phosphoglycerol kinase (PGK) promoter. In some embodiments, the promoter is selected from the group consisting of the chicken β-actin (CBA) promoter the EF-1 alpha promoter, and the EF-1 alpha short promoter. In some embodiments, the promoter comprises a sequence selected from any one of SEQ ID NO: 5-8, or a sequence at least 95% identical thereto.

In some embodiments, the AAV transfer cassettes described herein comprise an enhancer. The enhancer may be, for example, the CMV enhancer. In some embodiments, the enhancer comprises the sequence of SEQ ID NO: 9, or a sequence at least 95% identical thereto.

A non-limiting list of exemplary tissue-specific promoters and enhancers that may be used in the AAV transfer cassettes described herein includes: HMG-COA reductase promoter; sterol regulatory element 1 (SRE-1); phosphoenol pyruvate carboxy kinase (PEPCK) promoter; human C-reactive protein (CRP) promoter; human glucokinase promoter; cholesterol 7-alpha hydroylase (CYP-7) promoter; beta-galactosidase alpha-2,6 sialyltransferase promoter; insulin-like growth factor binding protein (IGFBP-1) promoter; aldolase B promoter; human transferrin promoter; collagen type I promoter; prostatic acid phosphatase (PAP) promoter; prostatic secretory protein of 94 (PSP 94) promoter; prostate specific antigen complex promoter; human glandular kallikrein gene promoter (hgt-1); the myocyte-specific enhancer binding factor MEF-2; muscle creatine kinase promoter; pancreatitis associated protein promoter (PAP); elastase 1 transcriptional enhancer; pancreas specific amylase and elastase enhancer promoter; pancreatic cholesterol esterase gene promoter; uteroglobin promoter; cholesterol side-chain cleavage (SCC) promoter; gamma-gamma enolase (neuron-specific enolase, NSE) promoter; neurofilament heavy chain (NF-H) promoter; human CGL-1/granzyme B promoter; the terminal deoxy transferase (TdT), lambda 5, VpreB, and Ick (lymphocyte specific tyrosine protein kinase p561ck) promoter; the humans CD2 promoter and its 3′ transcriptional enhancer; the human NK and T cell specific activation (NKG5) promoter; pp60c-src tyrosine kinase promoter; organ-specific neoantigens (OSNs), mw 40 kDa (p40) promoter; colon specific antigen-P promoter; human alpha-lactalbumin promoter; phosphoeholpyruvate carboxykinase (PEPCK) promoter, HER2/neu promoter, casein promoter, IgG promoter, Chorionic Embryonic Antigen promoter, elastase promoter, porphobilinogen deaminase promoter, insulin promoter, growth hormone factor promoter, tyrosine hydroxylase promoter, albumin promoter, alphafetoprotein promoter, acetyl-choline receptor promoter, alcohol dehydrogenase promoter, alpha or beta globin promoter, T-cell receptor promoter, the osteocalcin promoter the IL-2 promoter, IL-2 receptor promoter, whey (wap) promoter, and the MHC Class II promoter.

### Transgene

The AAV transfer cassettes described herein comprise a transgene for expression in a target cell.

The transgene may be any heterologous nucleic acid sequence(s) of interest. Such nucleic acids may include nucleic acids encoding polypeptides, including therapeutic (e.g., for medical or veterinary uses) or immunogenic (e.g., for vaccines) polypeptides or RNAs. Alternatively, the nucleic acid may encode an antisense nucleic acid, a ribozyme, RNAs that effect spliceosome-mediated/ram-splicing, interfering RNAs (RNAi) including siRNA, shRNA or miRNA that mediate gene silencing, and other non-translated RNAs. In some embodiments, the nucleic acid sequence may direct gene editing. For example, the nucleic acid may encode a gene-editing molecule such as a guide RNA or a nuclease. In some embodiments, the nucleic acid may encode a zinc-finger nuclease, a homing endonuclease, a TALEN (transcription activator-like effector nuclease), a NgAgo (agronaute endonuclease), a SGN (structure-guided endonuclease), or a RGN (RNA-guided nuclease) such as a Cas9 nuclease or a Cpf1 nuclease. In some embodiments, the nucleic acid may share homology with and recombine with a locus on a host chromosome. This approach can be utilized, for example, to correct a genetic defect in the host cell.

The virus vectors according to the present disclosure provide a means for delivering transgenes into a broad range of cells, including dividing and non-dividing cells. The virus vectors can be employed to deliver a transgene to a cell in vitro, e.g., to produce a polypeptide in vitro or for ex vivo gene therapy. The virus vectors are additionally useful in a method of delivering a transgene to a subject in need thereof, e.g., to express an immunogenic or therapeutic polypeptide or a functional RNA. In this manner, the polypeptide or functional RNA can be produced in vivo in the subject. The subject can be in need of the polypeptide because the subject has a deficiency of the polypeptide. Further, the method can be practiced because the production of the polypeptide or functional RNA in the subject may impart some beneficial effect. As used herein, the term “functional RNA” refers to any non-coding RNA sequence that has one or more functions in a cell, such as those described in the preceding paragraph.

The virus vectors can also be used to deliver nucleic acids for the production of a polypeptide of interest or functional RNA in cultured cells or in a subject (e.g., using the subject as a bioreactor to produce the polypeptide or to observe the effects of the functional RNA on the subject, for example, in connection with screening methods).

In general, the virus vectors of the present disclosure can be employed to deliver a transgene encoding a polypeptide or functional RNA to treat and/or prevent any disease state for which it is beneficial to deliver a therapeutic polypeptide or functional RNA.

In some embodiments, the transgene is useful for treating NPC1. In some embodiments, the transgene encodes the NPC1 protein. The NPC1 protein may be, for example, the human NPC1 protein. In some embodiments, the NPC1 protein has a sequence that is at least 90% identical, at least 95% identical, or at least 98% identical to the sequence of the human NPC1 protein. In some embodiments, the NPC1 protein comprises one or more of the single amino acid changes listed in Table 3 (numbering based on SEQ ID NO: 1). In some embodiments, the NPC1 protein comprises one or more of the amino acid changes listed in Table 3 (numbering based on SEQ ID NO: 20). In some embodiments, the NPC1 protein is a truncated form of the human NPC1 protein. In some embodiments, the NPC1 protein comprises the sequence of SEQ ID NO: 1, or a sequence at least 90% identical, at least 95% identical, at least 96% identical, at least 97% identical, at least 98% identical, or at least 99% identical thereto. In some embodiments, the NPC1 protein comprises the sequence of SEQ ID NO: 20, or a sequence at least 90% identical, at least 95% identical, at least 96% identical, at least 97% identical, at least 98% identical, or at least 99% identical thereto. In some embodiments, the NPC1 protein comprises the sequence of SEQ ID NO: 1 or 20, with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acid changes relative thereto. In some embodiments, the NPC1 protein comprises the sequence of SEQ ID NO: 1 or 20, with one or more of the amino acid changes listed in Table 3. In some embodiments, the transgene encodes the amino acid sequence of SEQ ID NO: 1. In some embodiments, the transgene encodes the amino acid sequence of SEQ ID NO: 20.

In some embodiments, the transgene comprises the sequence of SEQ ID NO: 2, or a sequence at least 90% identical, at least 95% identical, at least 96% identical, at least 97% identical, at least 98% identical, or at least 99% identical thereto. In some embodiments, the transgene comprises the sequence of SEQ ID NO: 2, or a sequence with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more nucleic acid changes relative thereto.

TABLE 3
NPC1 Variant Sequences
Position numbering based on SEQ ID NO: 1 or SEQ ID NO: 20.

| Position | Mutation |
|---|---|
| 63 | C→R |
| 74 | C→Y |
| 92 | Q→R |
| 113 | C→R |
| 137 | T→M |
| 151 | S→G |
| 166 | P→S |
| 177 | C→G |
| 177 | C→Y |
| 215 | H→R |
| 222 | N→S |
| 231 | V→G |
| 237 | P→S |
| 242 | D→H |
| 242 | D→N |
| 247 | C→Y |
| 248 | G→V |
| 272 | M→R |
| 333 | G→D |
| 372 | R→W |
| 378 | V→A |
| 380 | L→F |
| 381 | W→C |
| 388 | A→P |
| 389 | R→C |
| 401 | P→T |
| 404 | R→P |
| 404 | R→Q |
| 404 | R→W |
| 433 | P→L |
| 434 | P→L |
| 434 | P→S |
| 451 | E→K |
| 472 | L→P |
| 473 | S→P |
| 474 | P→L |
| 479 | C→Y |
| 509 | Y→S |
| 510 | H→P |
| 511 | T→M |
| 512 | H→R |
| 518 | R→Q |
| 518 | R→W |
| 521 | A→S |
| 537 | F→L |
| 543 | P→L |
| 574 | T→K |
| 576 | K→R |
| 605 | A→V |
| 612 | E→D |
| 615 | R→C |
| 615 | R→L |
| 631 | M→R |
| 640 | G→R |
| 642 | M→I |
| 652 | S→W |
| 660 | G→S |
| 664 | V→M |
| 666 | S→N |
| 670 | C→W |
| 673 | G→V |
| 684 | L→F |
| 691 | P→L |
| 695 | L→V |
| 700 | D→N |
| 703 | F→S |
| 724 | L→P |
| 727 | V→F |
| 734 | S→I |
| 742 | E→K |
| 745 | A→E |
| 754 | M→K |
| 757 | V→A |
| 763 | F→L |
| 767 | A→V |
| 775 | Q→P |
| 789 | R→C |
| 789 | R→G |
| 825 | Y→C |
| 849 | S→I |
| 858 | I→V |
| 862 | Q→L |
| 865 | S→L |
| 871 | Y→C |
| 873 | V→A |
| 874 | D→V |
| 888 | P→S |
| 889 | V→M |
| 890 | Y→C |
| 899 | Y→D |
| 910 | G→S |
| 917 | D→Y |
| 926 | A→T |
| 927 | A→V |
| 928 | Q→P |
| 929 | L→P |
| 934 | R→Q |
| 940 | S→L |
| 942 | W→C |
| 943 | I→M |
| 944 | D→N |
| 945 | D→N |
| 948 | D→H |
| 948 | D→N |
| 948 | D→Y |
| 950 | V→M |
| 954 | S→L |
| 956 | C→Y |
| 958 | R→L |
| 958 | R→Q |
| 959 | V→E |
| 961-966 | NITDQF→S |
| 961 | N→S |
| 968 | N→S |
| 971 | V→G |
| 976 | C→R |
| 978 | R→C |
| 986 | G→S |
| 992 | G→A |
| 992 | G→R |
| 992 | G→W |
| 996 | M→R |
| 1004 | S→L |
| 1007 | P→A |
| 1012 | G→D |
| 1015 | G→V |
| 1016 | H→R |
| 1023 | V→G |
| 1034 | G→R |
| 1035 | A→V |
| 1036 | T→K |
| 1036 | T→M |
| 1049 | A→V |
| 1054 | A→T |
| 1059 | R→Q |
| 1061 | I→T |
| 1062 | A→V |
| 1066 | T→N |
| 1087 | F→L |
| 1088 | Y→C |
| 1089 | E→K |
| 1094 | I→T |
| 1097 | D→N |
| 1137 | N→I |
| 1140 | G→V |
| 1142 | M→T |
| 1150 | N→K |
| 1156 | N→I |
| 1156 | N→S |
| 1165 | V→M |
| 1167 | F→L |
| 1168 | C→Y |
| 1174 | A→V |
| 1186 | R→H |
| 1189 | E→G |
| 1205 | T→K |
| 1205 | T→R |
| 1212 | V→L |
| 1213 | L→F |
| 1213 | L→V |
| 1216 | A→V |
| 1220 | I→T |
| 1224 | F→L |
| 1236 | G→E |
| 1240 | G→R |
| 1249 | S→G |
| 1266 | R→Q |

### Polyadenylation (PolyA) Signal

Polyadenylation signals are nucleotide sequences found in nearly all mammalian genes and control the addition of a string of approximately 200 adenosine residues (the poly(A) tail) to the 3′ end of the gene transcript. The poly(A) tail contributes to mRNA stability, and mRNAs lacking the poly(A) tail are rapidly degraded. There is also evidence that the presence of the poly(A) tail positively contributes to the translatability of mRNA by affecting the initiation of translation.

In some embodiments, the AAV transfer cassettes of the disclosure comprise a polyadenylation signal. The polyadenylation signal may be selected from the polyadenylation signal of simian virus 40 (SV40), α-globin, β-globin, human collagen, human growth hormone (hGH), polyoma virus, human growth hormone (hGH) and bovine growth hormone (bGH). In some embodiments, the polyadenylation signal is the SV40 polyadenylation signal. In some embodiments, the polyadenylation signal is the rBG polyadenylation signal. In some embodiments, the polyadenylation signal comprises the sequence of SEQ ID NO: 12 or SEQ ID NO: 13. In some embodiments, the polyadenylation signal comprises a sequence at least 90%, at least 95%, at least 96%, at least 97%, at least 98%, or at least 99% identical to the sequence of SEQ ID NO: 12 or SEQ ID NO: 13.

### Stuffer Sequences

AAV vectors typically accept inserts of DNA having a defined size range, generally in the range of about 4 kb to about 5.2 kb, or slightly more. Thus, for shorter transgene sequences, it may be necessary to include additional nucleic acids in order to achieve the required length which is acceptable for the AAV vector. Accordingly, in some embodiments, the AAV transfer cassettes of the disclosure may comprise a suffer sequence. The stuffer sequence may be for example, a sequence between 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, to 4,500-5,000 nucleotides in length. The stuffer sequence can be located in the cassette at any desired position such that it does not prevent a function or activity of the vector.

### Intronic Sequences

In some embodiments, the AAV transfer cassettes of the disclosure may comprise an intronic sequence. Inclusion of an intronic sequence may enhance expression compared with expression in the absence of the intronic sequence. In some the intronic sequence can increase gene expression without functioning as a binding site for transcription factors. For example, the intronic sequence can increase transcript levels by affecting the rate of transcription, nuclear export, and transcript stability. In some embodiments, the intronic sequence increases the efficiency of mRNA translation.

In some embodiments, the intronic sequence is a hybrid or chimeric sequence. In some embodiments, the intronic sequence is isolated or derived from an intronic sequence of one or more of SV40, β-globin, chicken beta-actin, minute virus of mice (MVM), factor IX, and/or human IgG (heavy or light chain). In some embodiments, the intronic sequence is isolated or derived from SV40. In some embodiments, the intronic sequence is chimeric. In some embodiments, the intronic sequence comprises the sequence of SEQ ID NO: 10 or SEQ ID NO: 11, or a sequence that is at least 90%, at least 95%, at least 96%, at least 97%, at least 98%, or at least 99% identical thereto.

The intronic sequence may be located anywhere in the transfer cassette where it doesn't interfere with production of the AAV vector. For example, in some embodiments, the intronic sequence may be located between the promoter and the transgene.

### AAV Transfer Cassettes

In some embodiments, an adeno-associated virus (AAV) transfer cassette comprises a 5′ inverted terminal repeat (ITR), a promoter, a transgene, a polyadenylation signal, and a 3′ ITR. In some embodiments, the transfer cassette comprises an intronic sequence, such as an intronic sequence between the promoter and the transgene. In some embodiments, the transgene encodes the NPC1 protein. In some embodiments, the AAV transfer cassette comprises an enhancer. In some embodiments, the AAV transfer cassette comprises an intronic sequence. In some embodiments, the 5′ ITR comprises the sequence of SEQ ID NO: 3 and the 3′ ITR comprises the sequence of SEQ ID NO: 4. In some embodiments, the enhancer comprises the sequence of SEQ ID NO: 9. In some embodiments, the promoter comprises the sequence of any one of SEQ ID NO: 5-8. In some embodiments, the intronic sequence comprises the sequence of SEQ ID NO: 10 or 11. In some embodiments, the transgene comprises the sequence of SEQ ID NO: 2. In some embodiments, the polyA signal comprises the sequence of SEQ ID NO: 12 or 13. In some embodiments, the AAV transfer cassette comprises the sequence of any one of SEQ ID NO: 14-19. In some embodiments, the AAV transfer cassette comprises the sequence of SEQ ID NO: 14.

In some embodiments, an AAV transfer cassette comprises a 5′ ITR, a CBA promoter, a SV40 intron, a transgene encoding the NPC1 protein, a SV40 polyadenylation signal, and a 3′ ITR. In some embodiments, an AAV transfer cassette comprises a 5′ ITR, a GUSB240 promoter, a chimeric intron, a transgene encoding the NPC1 protein, a rBG polyadenylation signal, and a 3′ ITR. In some embodiments, an AAV transfer cassette comprises a 5′ ITR, a GUSB379 promoter, a SV40 intron, a transgene encoding the NPC1 protein, a rBG polyadenylation signal, and a 3′ ITR. In some embodiments, an AAV transfer cassette comprises a 5′ ITR, a GUSB240 promoter, a chimeric intron, a transgene encoding the NPC1 protein, a SV40 polyadenylation signal, and a 3′ ITR. In some embodiments, an AAV transfer cassette comprises a 5′ ITR, a GUSB240 promoter, a SV40 intron, a transgene encoding the NPC1 protein, a SV40 polyadenylation signal, and a 3′ ITR. In some embodiments, an AAV transfer cassette comprises a 5′ ITR, a CMV enhancer, a HSVTK promoter, a transgene encoding the NPC1 protein, a rBG polyadenylation signal, and a 3′ ITR.

In some embodiments, an AAV transfer cassette comprises a 5′ ITR comprising the sequence of SEQ ID NO: 3, a CBA promoter comprising the sequence of SEQ ID NO: 5, a SV40 intron comprising the sequence of SEQ ID NO: 10, a transgene encoding the NPC1 protein (SEQ ID NO: 1), a SV40 polyadenylation signal comprising SEQ ID NO: 12, and a 3′ ITR comprising the sequence of SEQ ID NO: 4.

In some embodiments, an AAV transfer cassette comprises a 5′ ITR comprising the sequence of SEQ ID NO: 3, a GUSB240 promoter comprising the sequence of SEQ ID NO: 6, a chimeric intron comprising SEQ ID NO: 11, a transgene encoding the NPC1 protein (SEQ ID NO: 1), a rBG polyadenylation signal comprising SEQ ID NO: 13, and a 3′ ITR comprising the sequence of SEQ ID NO: 4.

In some embodiments, an AAV transfer cassette comprises a 5′ ITR comprising the sequence of SEQ ID NO: 3, a GUSB379 promoter comprising SEQ ID NO: 6, a SV40 intron comprising the sequence of SEQ ID NO: 10, a transgene encoding the NPC1 protein (SEQ ID NO: 1), a rBG polyadenylation signal comprising SEQ ID NO: 13, and a 3′ ITR comprising the sequence of SEQ ID NO: 4.

In some embodiments, an AAV transfer cassette comprises a 5′ ITR comprising the sequence of SEQ ID NO: 3, a GUSB240 promoter comprising SEQ ID NO: 7, a chimeric intron comprising the sequence of SEQ ID NO: 11, a transgene encoding the NPC1 protein (SEQ ID NO: 1), a SV40 polyadenylation signal comprising SEQ ID NO: 12, and a 3′ ITR comprising the sequence of SEQ ID NO: 4.

In some embodiments, an AAV transfer cassette comprises a 5′ ITR comprising the sequence of SEQ ID NO: 3, a GUSB240 promoter comprising SEQ ID NO: 6, a SV40 intron comprising the sequence of SEQ ID NO: 10, a transgene encoding the NPC1 protein (SEQ ID NO: 1), a SV40 polyadenylation signal comprising SEQ ID NO: 12, and a 3′ ITR comprising the sequence of SEQ ID NO: 4.

In some embodiments, an AAV transfer cassette comprises a 5′ ITR comprising the sequence of SEQ ID NO: 3, a CMV enhancer, a HSVTK promoter comprising SEQ ID NO: 8, a transgene encoding the NPC1 protein (SEQ ID NO: 1), a rBG polyadenylation signal comprising SEQ ID NO: 13, and a 3′ ITR comprising the sequence of SEQ ID NO: 4.

In some embodiments, a nucleic acid comprises an AAV transfer cassette. In some embodiments, a nucleic acid comprises a transgene, wherein the transgene encodes the amino acid sequence of SEQ ID NO: 1. In some embodiments, a nucleic acid comprises a transgene, wherein the transgene encodes the amino acid sequence of SEQ ID NO: 20. In some embodiments, a nucleic acid comprises, from 5′ to 3′, a 5′ inverted terminal repeat (ITR); a promoter; a transgene; a polyadenylation signal; and a 3′ ITR; wherein the transgene encodes the amino acid sequence of SEQ ID NO: 1 or SEQ ID NO:20. In some embodiments, a nucleic acid comprises, from 5′ to 3′, a 5′ inverted terminal repeat (ITR); a promoter; an intronic sequence; a transgene; a polyadenylation signal; and a 3′ ITR; wherein the transgene encodes the amino acid sequence of SEQ ID NO: 1 or SEQ ID NO:20. In some embodiments, a nucleic acid comprises, from 5′ to 3′, a 5′ inverted terminal repeat (ITR); a chicken beta-actin promoter; an intronic sequence; a transgene; a polyadenylation signal; and a 3′ ITR; wherein the transgene encodes the amino acid sequence of SEQ ID NO: 1 or SEQ ID NO:20.

The AAV transfer cassettes described herein may be incorporated into a plasmid or a bacmid using standard molecular biology techniques. The plasmid or bacmid may further comprise one or more genetic elements used during production of AAV, including, for example, AAV rep and cap genes, and helper virus protein sequences.

### AAV Production Methods

The AAV transfer cassettes, and nucleic acids (e.g., plasmids) comprising the AAV transfer cassettes described herein may be used to produce recombinant AAV vectors.

In some embodiments, a method for producing a recombinant AAV vector comprises contacting an AAV producer cell (e.g., an HEK293 cell) with an AAV transfer cassette or nucleic acid (e.g., plasmid) of the disclosure. In some embodiments, the method further comprises contacting the AAV producer cell with one or more additional plasmids encoding, for example, AAV rep and cap genes, and helper virus protein sequences.

In some embodiments, a method for producing a recombinant AAV vector comprises contacting an AAV producer cell (e.g., an insect cell such as a Sf9 cell) with at least one insect cell-compatible nucleic acid comprising an AAV transfer cassette of the disclosure. An “insect cell-compatible” nucleic acid is any nucleic acid which may be transformed or transfected into an insect cell, and which may be recognized by the transcription and/or translation machinery of the cell. In some embodiments, the insect cell-compatible nucleic acid is a baculoviral nucleic acid. In some embodiments, the method further comprises maintaining the insect cell under conditions such that AAV is produced.

The recombinant AAV vectors produced may be of any serotype, for example AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV and Bovine AAV. In some embodiments, the recombinant AAV vectors produced may comprise a protein capsid comprising a capsid protein subunit, wherein the capsid protein subunit comprises one or more amino acid modifications (e.g., substitutions and/or deletions) compared to the native AAV capsid protein subunit. For example, the recombinant AAV vectors may be modified AAV vectors derived from AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV and Bovine AAV.

The recombinant AAV vectors may be used to transduce target cells with the transgene, for example by contacting the recombinant AAV vector with a target cell.

### Compositions

Also provided are compositions comprising an AAV transfer cassette, a plasmid, a cell, or a recombinant AAV vector of the disclosure. In some embodiments, the compositions may further comprise a pharmaceutically acceptable carrier or excipient.

### Methods of Treatment

The AAV vectors of the disclosure may be used to treat or prevent a disease, disorder, or other condition a subject in need thereof. The subject may be, for example a human or an animal. The human may be a pediatric subject, an adolescent subject, an adult subject, or a geriatric subject.

Thus, a further aspect of the disclosure is a method of administering the virus vector, virus particle and/or virus-like particle of the disclosure to a subject.

The present disclosure also provides a method of delivering a nucleic acid to a subject, comprising administering to the subject a virus vector and/or composition of this disclosure. Administration of the virus vectors, and/or compositions according to the present disclosure to a subject in need thereof can be by any means known in the art. Optionally, the virus vector and/or composition is delivered in an effective dose (e.g., a therapeutically effective dose) in a pharmaceutically acceptable carrier. In preferred embodiments, an effective amount of the virus vector and/or composition is delivered.

Dosages of the virus vector and/or virus-like particle to be administered to a subject depend upon the mode of administration, the disease or condition to be treated and/or prevented, the individual subject's condition, the particular virus vector or particle, and the nucleic acid to be delivered, and the like, and can be determined in a routine manner. In some embodiments, the dose of recombinant AAV is an effective dose. Exemplary effective doses may be, for example, a dose of at least about 10⁵, about 10⁶, about 10⁷, about 10⁸, about 10⁹, about 10¹⁰, about 10¹¹, about 10¹², about 10¹³, about 10¹⁴, about 10¹⁵ transducing units, optionally about 10⁸ to about 10¹³ transducing units. In some embodiments, an effective dose of recombinant AAV is a dose in the range of about 1×10¹¹ to about 1×10¹⁵ vector genomes per kilogram body weight of the subject. For example, the effective dose may be about 1×10¹¹, about 5×10¹¹, about 1×10¹², about 5×10¹², about 1×10¹³, about 5×10¹³, about 1×10¹⁴, about 5×10¹⁴, or about 1×10¹⁵ vector genomes per kilogram body weight of the subject.

In particular embodiments, more than one administration (e.g., two, three, four or more administrations) may be employed to achieve the desired level of gene expression over a period of various intervals, e.g., daily, weekly, monthly, yearly, etc.

Exemplary modes of administration include oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). Administration can also be to a tumor (e.g., in or near a tumor or a draining lymph node). The most suitable route in any given case will depend on the nature and severity of the condition being treated and/or prevented and on the nature of the particular AAV vector that is being used.

Delivery to a target tissue can also be achieved by delivering a depot comprising the virus vector and/or virus-like particle. As described herein, delivery of a “depot” refers to administration of a sustained-action formulation that allows slow release and/or gradual dissemination of the virus, so that the virus can act for longer periods than is possible with standard injections. In representative embodiments, a depot comprising the virus vector and/or virus-like particle is implanted into skeletal, cardiac and/or diaphragm muscle tissue or the tissue can be contacted with a film or other matrix comprising the virus vector and/or viral-like particle.

In some embodiments, a method for treating a subject in need thereof comprises administering to the subject an effective amount (e.g., a therapeutically effective amount) of an AAV transfer cassette, a plasmid, a cell, or a recombinant AAV of the disclosure. In some embodiments, the subject is a human subject. In some embodiments, the subject has NPC1.

### EXAMPLES

The following examples, which are included herein for illustration purposes only, are not intended to be limiting.

### Example 1: Preparation of a Recombinant AAV Vector in Mammalian Cells

Three plasmids are provided. The first plasmid comprises a transfer cassette comprising a transgene (SEQ ID NO: 2) encoding NPC1 flanked by two ITRs (SEQ ID NO: 3 and 4). The first plasmid comprises the sequence of any one of SEQ ID NO: 14-19. The second plasmid comprises sequences encoding the Rep and Cap proteins. The third plasmid comprises various “helper” factors required for AAV production (E4, E2a, and VA).

The three plasmids are transfected into viral production cells (e.g., HEK293) using an appropriate transfection reagent (e.g., Lipofectamine™). After incubation at 37° C. for a predetermined period of time, AAV particles are collected from the media or the cells are lysed to release the AAV particles. The AAV particles are then purified and titered using either quantitative PCR (qPCR) or droplet digital PCR (ddPCR) according to standard methods. The AAV particles may be stored at −80° C. for later use.

### Example 2: Preparation of a Recombinant AAV Vector in Insect Cells

A first recombinant baculoviral vector is provided. The first recombinant baculoviral vector comprises a nucleic acid comprising a transfer cassette comprising a transgene (SEQ ID NO: 2) encoding NPC1, wherein the transgene is flanked by two ITRs (SEQ ID NO: 3 and 4). The transfer cassette comprises the sequence of any one of SEQ ID NO: 14-19.

Insect cells (e.g., Sf9) are co-infected in suspension culture with the first recombinant baculoviral vector and a least one additional recombinant baculoviral vector comprising a nucleic acid encoding the AAV Rep and Cap proteins. After incubation at 28° C. for a predetermined period of time, AAV particles are collected from the media or the cells are lysed to release the AAV particles. The AAV particles are then purified and titered using either quantitative PCR (qPCR) or droplet digital PCR (ddPCR) according to standard methods. The AAV particles may be stored at −80° C. for later use.

### Example 3: In Vitro Potency Assay

To determine whether the AAV transfer cassettes described herein are able to rescue the NPC1 lysosomal phenotype in cultured cells, a recombinant AAV2 vector packaging a hNPC1 transfer cassette (SEQ ID NO: 14) was prepared in HEK293 cells using a triple-transfection protocol (See, e.g., Example 1). The AAV2-hNPC1 vector was then used to transduce wildtype U2OS cells (osteosarcoma), and U2OS cells which do not express NPC1 (NPC^{-/-}) in vitro at a multiplicity of infection (MOI) of either 5×10³ (5K) or 10×10³ (10K). Cells were then incubated at 37° C. in a 5% CO₂ atmosphere.

NPC1 cells exhibit a characteristic accumulation of cholesterol in lysosomes, which can be monitored by observing the size and number of lysosomes in a cell. In this assay, lysosomal phenotype was monitored by measuring accumulation of a fluorescent organelle dye, LysoTracker® (ThermoFisher Scientific©), in the cells. 72 hours after transduction with the AAV2-hNPC1 vector, 50 mM of LysoTracker® was added to the cells. After 2 hours, the cells were fixed and LysoTracker© fluorescence was measured.

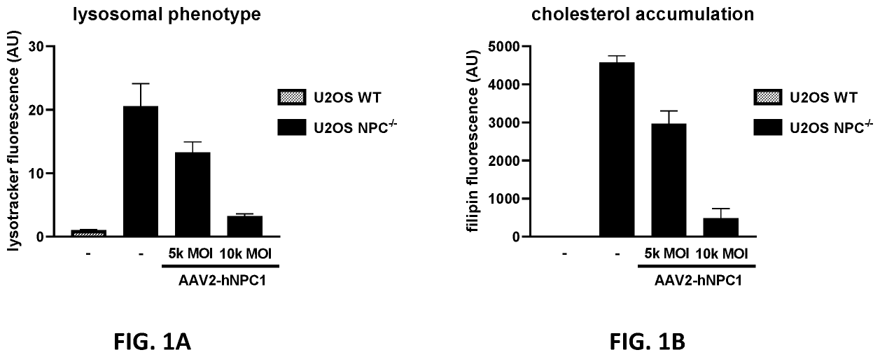

Results are shown in FIG. 1A. As expected, wildtype U2OS cells did not show significant accumulation of LysoTracker® fluorescence in lysosomes, whereas the NPC1^{-/-} cells did. Cells transduced with AAV2-hNPC1 at a MOI of either 5K or 10K had significantly reduced accumulation of LysoTracker® fluorescence in lysosomes.

In a separate assay, cells transduced with hNPC1 were fixed and stained using filipin, a histochemical stain for cholesterol. The filipin stain, derived from Streptomyces filipinensis, was purchased from Polysciences, and was used at a final concentration of 50 μg/mL. The cells were visualized using a Pico Automated Cell Imaging System (ImageXpress®), and filipin stain was quantified. Results are shown in FIG. 1B. As expected, wildtype U20S cells did not show significant cholesterol accumulation, whereas the NPC1^{-/-} cells did. Cells transduced with AAV2-hNPC1 at a MOI of either 5K or 10K had significantly reduced cholesterol accumulation.

Taken together, these data show that transduction of cells using AAV2-hNPC successfully rescued lysosomal phenotype in NPC1-deficient U20S cells.

### Example 4: In Vivo Potency Assay

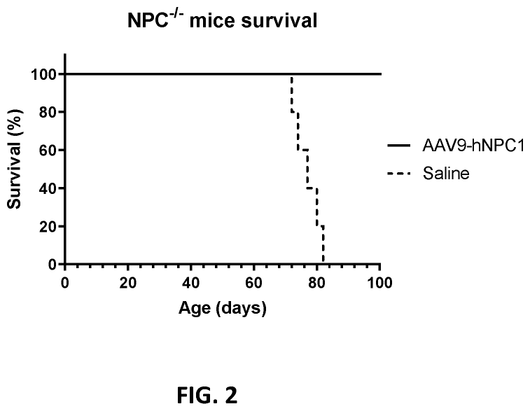

To determine whether the AAV transfer cassettes described herein are able to rescue the NPC1 phenotype in vivo, a recombinant AAV9 vector packaging a hNPC1 transfer cassette (SEQ ID NO: 14) was prepared in HEK293 cells using a triple-transfection protocol (See, e.g., Example 1). Mice deficient for NPC1 (i.e., NPC1^{-/-} mice) were injected intravenously at a dose of 3.0×10¹⁴ vector genomes per kilogram (vg/kg), by retro-orbital injection, with either saline or with the AAV9-hNPC1 vector around the age of 24-28 days. Results are shown in FIG. 2. All saline-treated mice died by the age of about 80 days. However, all AAV9-hNPC1-injected animals survived through the duration of experiment. The AAV9-hNPC1-injected mice were sacrificed around 100 days of age for analysis.

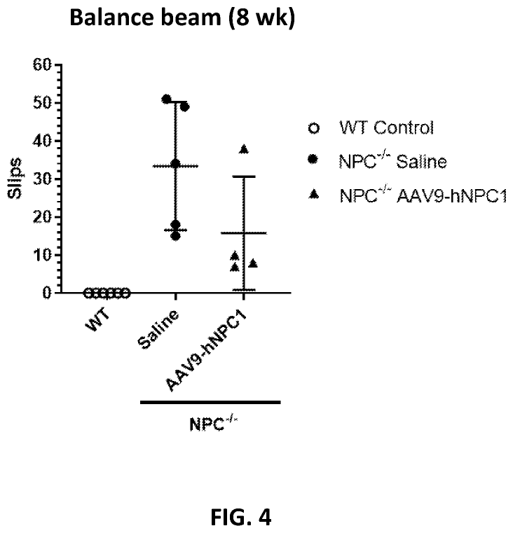

Mice were also challenged in a balance beam walking test, wherein number of slips were measured as mice walked across a balance beam. The test was performed at about 8 weeks (56 days) of age. As shown in FIG. 4, wildtype mice did not slip off the balance beam. Although there was no statistically significant difference in the number of slips between NPC1^{-/-} mice treated with AAV9-hNPC1 and saline-treated NPC1^{-/-} mice, the average number of slips observed in the AAV9-hNPC1 group was less.

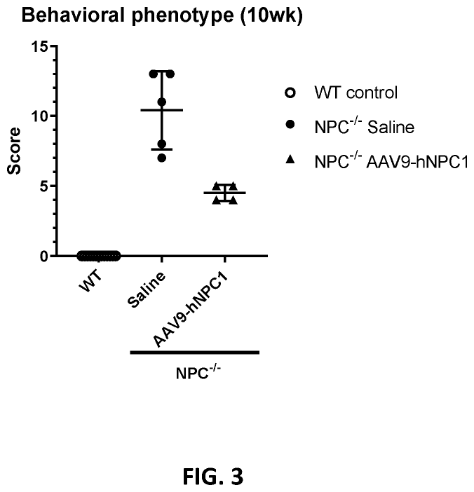

Behavioral phenotype score of the mice was also assessed at about 10 weeks (70 days) of age. The behavioral phenotype score is a composite score measuring various disease symptoms, including grooming, gait, kyphosis, ledge test, hindlimb clasp, and tremor. (See Alam et al, Sci Transl Med, 2016; Guyenet et al, J Vis Exp, 2010). As shown in FIG. 3, NPC1^{-/-} mice treated with AAV9-hNPC1 had a significantly reduced score as compared to saline-treated NPC1^{-/-} mice.

Taken together, these data demonstrate that AAV9-hNPC1 can at least partially rescue the disease phenotype of NPC1 deficient mice.

The foregoing is illustrative of the present invention, and is not to be construed as limiting thereof. The invention is defined by the following claims, with equivalents of the claims to be included therein.

### Numbered Embodiments

Notwithstanding the appended claims, the following numbered embodiments also form part of the instant disclosure.

1. An Adeno-Associated Virus (AAV) transfer cassette comprising, from 5′ to 3′: a 5′ inverted terminal repeat (ITR); a promoter; a transgene; a polyadenylation signal; and a 3′ ITR; wherein the transgene encodes the NPC1 protein.

2. The AAV transfer cassette of embodiment 1, wherein at least one of the 5′ ITR and the 3′ ITR is about 110 to about 160 nucleotides in length.

3. The AAV transfer cassette of embodiment 1 or 2, wherein the 5′ ITR is the same length as the 3′ ITR.

4. The AAV transfer cassette of embodiment 1 or 2, wherein the 5′ ITR and the 3′ ITR have different lengths.

5. The AAV transfer cassette of any one of embodiments 1-4, wherein at least one of the 5′ ITR and the 3′ ITR is isolated or derived from the genome of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.

6. The AAV transfer cassette of embodiment 1, wherein the 5′ ITR comprises the sequence of SEQ ID NO: 3.

7. The AAV transfer cassette of embodiment 1, wherein the 3′ ITR comprises the sequence of SEQ ID NO: 4.

8. The AAV transfer cassette of any one of embodiments 1-7, wherein the promoter is a constitutive promoter.

9. The AAV transfer cassette of any one of embodiments 1-7, wherein the promoter is an inducible promoter.

10. The AAV transfer cassette of any one of embodiments 1-9, wherein the promoter is a tissue-specific promoter.

11. The AAV transfer cassette of any one of embodiments 1-7, wherein the promoter is selected from the group consisting of the CBA promoter, the GUSB240 promoter, the GUSB379 promoter, the HSVTK promoter, the CMV promoter, the SV40 early promoter, the SV40 late promoter, the metallothionein promoter, the murine mammary tumor virus (MMTV) promoter, the Rous sarcoma virus (RSV) promoter, the polyhedrin promoter, the chicken β-actin (CBA) promoter, the EF-1 alpha promoter, the dihydrofolate reductase (DHFR) promoter, and the phosphoglycerol kinase (PGK) promoter.

12. The AAV transfer cassette of embodiment 11, wherein the promoter is selected from the group consisting of the CBA promoter, the GUSB240 promoter, the GUSB379 promoter, and the HSVTK promoter.

13. The AAV transfer cassette of any one of embodiments 1-7, wherein the promoter comprises a sequence at least 95% or 100% identical to any one of SEQ ID NO: 5, SEQ ID NO: 6, SEQ ID NO: 7, or SEQ ID NO: 8.

14. The AAV transfer cassette of any one of embodiments 1-13, wherein the NPC1 protein is the human NPC1 protein.

15. The AAV transfer cassette of any one of embodiments 1-13, wherein the NPC1 protein has a sequence that is at least 90% identical to the sequence of the human NPC1 protein.

16. The AAV transfer cassette of embodiment 15, wherein the NPC1 protein has a sequence that is at least 95% identical to the sequence of the human NPC1 protein.

17. The AAV transfer cassette of embodiment 16, wherein the NPC1 protein has a sequence that is at least 98% identical to the sequence of the human NPC1 protein.

18. The AAV transfer cassette of any one of embodiments 1-13, wherein the NPC1 protein comprises the sequence of SEQ ID NO: 1.

19. The AAV transfer cassette of any one of embodiments 1-13, wherein the transgene comprises the sequence of SEQ ID NO: 2.

20. The AAV transfer cassette of any one of embodiments 1-18, wherein the polyadenylation signal is selected from simian virus 40 (SV40), rBG, α-globin, β-globin, human collagen, human growth hormone (hGH), polyoma virus, human growth hormone (hGH) and bovine growth hormone (bGH).

21. The AAV transfer cassette of embodiment 20, wherein the polyadenylation signal is the SV40 polyadenylation signal.

22. The AAV transfer cassette of embodiment 20, wherein the polyadenylation signal is the rBG polyadenylation signal.

23. The AAV transfer cassette of any one of embodiments 1-19, wherein the polyadenylation signal comprises the sequence at least 95% or 100% identical to SEQ ID NO: 12 or to SEQ ID NO: 13.

24. The AAV transfer cassette of any one of embodiments 1-23, wherein the cassette further comprises an enhancer.

25. The AAV transfer cassette of embodiment 24, wherein the enhancer is the CMV enhancer.

26. The AAV transfer cassette of embodiment 24, wherein the enhancer comprises the sequence of SEQ ID NO: 9, or a sequence at least 95% identical thereto.

27. The AAV transfer cassette of any one of embodiments 1-26, wherein the cassette further comprises an intronic sequence.

28. The AAV transfer cassette of embodiment 27, wherein the intronic sequence is a chimeric sequence.

29. The AAV transfer cassette of embodiment 27, wherein the intronic sequence is a hybrid sequence.

30. The AAV transfer cassette of embodiment 27, wherein the intronic sequence comprises sequences isolated or derived from SV40.

31. The AAV transfer cassette of embodiment 27, wherein the intronic sequence comprises the sequence of any one of SEQ ID NO: 10-11.

32. The AAV transfer cassette of embodiment 1, wherein the AAV transfer cassette comprises the sequence of any one of SEQ ID NO: 14-19.

33. A plasmid comprising the AAV transfer cassette of any one of embodiments 1-31.

34. A cell comprising the AAV transfer cassette of any one of embodiments 1-32 or the plasmid of embodiment 33.

35. A recombinant AAV vector comprising a protein capsid and a nucleic acid encapsidated by the protein capsid: wherein the nucleic acid comprises a transfer cassette comprising, from 5′ to 3′: a 5′ inverted terminal repeat (ITR); a promoter; a transgene; a polyadenylation signal; and a 3′ ITR; wherein the transgene encodes the NPC1 protein.

36. The recombinant AAV vector of embodiment 35, wherein at least one of the 5′ ITR and the 3′ ITR is about 110 to about 160 nucleotides in length.

37. The recombinant AAV vector of embodiment 36 or 36, wherein the 5′ ITR is the same length as the 3′ ITR.

38. The recombinant AAV vector of embodiment 35 or 36, wherein the 5′ ITR and the 3′ ITR have different lengths.

39. The recombinant AAV vector of any one of embodiments 35-38, wherein at least one of the 5′ ITR and the 3′ ITR is isolated or derived from the genome of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.

40. The recombinant AAV vector of embodiment 35, wherein the 5′ ITR comprises the sequence of SEQ ID NO: 3.

41. The recombinant AAV vector of embodiment 35, wherein the 3′ ITR comprises the sequence of SEQ ID NO: 4.

42. The recombinant AAV vector of any one of embodiments 35-41, wherein the promoter is a constitutive promoter.

43. The recombinant AAV vector of any one of embodiments 35-41, wherein the promoter is an inducible promoter.

44. The recombinant AAV vector of any one of embodiments 35-41, wherein the promoter is a tissue-specific promoter.

45. The recombinant AAV vector of any one of embodiments 35-41, wherein the promoter is selected from the group consisting of the CBA promoter, the GUSB240 promoter, the GUSB379 promoter, the HSVTK promoter, the CMV promoter, the SV40 early promoter, the SV40 late promoter, the metallothionein promoter, the murine mammary tumor virus (MMTV) promoter, the Rous sarcoma virus (RSV) promoter, the polyhedrin promoter, the chicken β-actin (CBA) promoter, the EF-1 alpha promoter, the dihydrofolate reductase (DHFR) promoter, and the phosphoglycerol kinase (PGK) promoter.

46. The recombinant AAV vector of embodiment 45, wherein the promoter is selected from the group consisting of the CBA promoter, the GUSB240 promoter, the GUSB379 promoter, and the HSVTK promoter.

47. The recombinant AAV vector of any one of embodiments 35-41, wherein the promoter comprises a sequence at least 95% or 100% identical to any one of SEQ ID NO: 5, SEQ ID NO: 6, SEQ ID NO: 7, or SEQ ID NO: 8.

48. The recombinant AAV vector of any one of embodiments 35-47, wherein the NPC1 protein is the human NPC1 protein.

49. The recombinant AAV vector of any one of embodiments 35-48, wherein the NPC1 protein has a sequence that is at least 90% identical to the sequence of the human NPC1 protein.

50. The recombinant AAV vector of embodiment 49, wherein the NPC1 protein has a sequence that is at least 95% identical to the sequence of the human NPC1 protein.

51. The recombinant AAV vector of embodiment 50, wherein the NPC1 protein has a sequence that is at least 98% identical to the sequence of the human NPC1 protein.

52. The recombinant AAV vector of any one of embodiments 35-48, wherein the NPC1 protein comprises the sequence of SEQ ID NO: 1.

53. The recombinant AAV vector of any one of embodiments 35-48, wherein the transgene comprises the sequence of SEQ ID NO: 2.

54. The recombinant AAV vector of any one of embodiments 35-53, wherein the polyadenylation signal is selected from simian virus 40 (SV40), rBG, α-globin, β-globin, human collagen, human growth hormone (hGH), polyoma virus, human growth hormone (hGH) and bovine growth hormone (bGH).

55. The recombinant AAV vector of embodiment 54, wherein the polyadenylation signal is the SV40 polyadenylation signal.

56. The recombinant AAV vector of embodiment 54, wherein the polyadenylation signal is the rBG polyadenylation signal.

57. The recombinant AAV vector of any one of embodiments 35-53, wherein the polyadenylation signal comprises the sequence at least 95% or 100% identical to SEQ ID NO: 12 or to SEQ ID NO: 13.

58. The recombinant AAV vector of any one of embodiments 35-57, wherein the cassette further comprises an enhancer.

59. The recombinant AAV vector of embodiment 58, wherein the enhancer is the CMV enhancer.

60. The recombinant AAV vector of embodiment 58, wherein the enhancer comprises the sequence of SEQ ID NO: 9, or a sequence at least 95% identical thereto.

61. The recombinant AAV vector of any one of embodiments 35-60, wherein the cassette further comprises an intronic sequence.

62. The recombinant AAV vector of embodiment 61, wherein the intronic sequence is a chimeric sequence.

63. The recombinant AAV vector of embodiment 61, wherein the intronic sequence is a hybrid sequence.

64. The recombinant AAV vector of embodiment 61, wherein the intronic sequence comprises sequences isolated or derived from SV40.

65. The recombinant AAV vector of embodiment 61, wherein the intronic sequence comprises the sequence of any one of SEQ ID NO: 10-11.

66. The recombinant AAV vector of embodiment 35, wherein the AAV transfer cassette comprises the sequence of any one of SEQ ID NO: 14-19.

67. The recombinant AAV vector of embodiment 35, wherein the AAV transfer cassette comprises the sequence of SEQ ID NO: 14.

68. The recombinant AAV vector of any one of embodiments 35-67, wherein the protein capsid comprises a capsid protein subunit from an AAV of any one of the following serotypes: AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.

69. The recombinant AAV vector of any one of embodiments 35-67, wherein the protein capsid comprises a capsid protein subunit that has one or more amino acid mutations relative to a capsid protein subunit of any one of the following AAV serotypes: AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.

70. A method of producing a recombinant AAV vector, the method comprising contacting an AAV producer cell with the AAV transfer cassette of any one of embodiments 1-32 or the plasmid of embodiment 33.

71. A recombinant AAV vector produced by the method of embodiment 35.

72. The recombinant AAV vector of embodiment 71, wherein the vector is of a serotype selected from AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV and Bovine AAV.

73. A composition comprising the AAV transfer cassette of any one of embodiments 1-32, the plasmid of embodiment 33, the cell of embodiment 34, or the recombinant AAV vector of any one of embodiments 35-69, 71, or 72.

74. A method for treating a subject in need thereof comprising administering to the subject an effective amount of the AAV transfer cassette of any one of embodiments 1-32, the plasmid of embodiment 33, the cell of embodiment 34, or the recombinant AAV vector of any one of embodiments 35-68, 70 or 71.

75. The method of embodiment 74, wherein the subject has NPC1.

76. The method of embodiment 74 or 75, wherein the subject is a human subject.

77. A nucleic acid encoding comprising, from 5′ to 3′, a 5′ inverted terminal repeat (ITR); a promoter; a transgene; a polyadenylation signal; and a 3′ ITR; wherein the transgene encodes the amino acid sequence of SEQ ID NO: 1 or SEQ ID NO:20.

78. An Adeno-Associated Virus (AAV) transfer cassette comprising, from 5′ to 3′: a 5′ inverted terminal repeat (ITR); a chicken beta-actin promoter; a transgene; a polyadenylation signal; and a 3′ ITR; wherein the transfer cassette comprises an intronic sequence; wherein the transgene encodes the NPC1 protein.

79. The AAV transfer cassette of embodiment 78, wherein the intronic sequence is located between the promoter and the transgene.

80. The AAV transfer cassette of embodiment 78 or 79, wherein the 5′ ITR comprises the sequence of SEQ ID NO: 3.

81. The AAV transfer cassette of any one of embodiments 78-80, wherein the 3′ ITR comprises the sequence of SEQ ID NO: 4.

82. The AAV transfer cassette of any one of embodiments 78-81, wherein the promoter comprises the sequence of SEQ ID NO: 5.

83. The AAV transfer cassette of any one of embodiments 78-82, wherein the intronic sequence is an SV40 intron.

84. The AAV transfer cassette of any one of embodiments 78-82, wherein the intronic sequence comprises the sequence of SEQ ID NO: 10.

85. The AAV transfer cassette of any one of embodiments 78-84, wherein the NPC1 protein is the human NPC1 protein.

86. The AAV transfer cassette of any one of embodiments 78-84, wherein the NPC1 protein comprises the sequence of SEQ ID NO: 1.

87. The AAV transfer cassette of any one of embodiments 78-84, wherein the transgene comprises the sequence of SEQ ID NO: 2.

88. The AAV transfer cassette of any one of embodiments 78-87, wherein the polyadenylation signal is the SV40 polyadenylation signal.

89. The AAV transfer cassette of any one of embodiments 78-87, wherein the polyadenylation signal comprises the sequence of SEQ ID NO: 12.

90. The AAV transfer cassette of any one of embodiments 78-89, wherein the cassette comprises an enhancer.

91. The AAV transfer cassette of embodiment 78, wherein the AAV transfer cassette comprises the sequence of SEQ ID NO: 14.

92. The AAV transfer cassette of embodiment 78, wherein the AAV transfer cassette comprises the sequence of any one of SEQ ID NO: 15-19.

93. A plasmid comprising the AAV transfer cassette of any one of embodiments 78-92.

94. A nucleic acid comprising, from 5′ to 3′, a 5′ inverted terminal repeat (ITR); a promoter; a transgene; a polyadenylation signal; and a 3′ ITR; wherein the nucleic acid comprises an intronic sequence; wherein the transgene encodes the amino acid sequence of SEQ ID NO: 1.

95. The nucleic acid of embodiment 94, wherein the intronic sequence is located between the promoter and the transgene.

96. A cell comprising the AAV transfer cassette of any one of embodiments 78-92, the plasmid of embodiment 93, or the nucleic acid of embodiment 94 or 95.

97. A recombinant AAV vector comprising a protein capsid and a nucleic acid encapsidated by the protein capsid: wherein the nucleic acid comprises the AAV transfer cassette of any one of embodiments 78-92.

98. The recombinant AAV vector of embodiment 97, wherein the protein capsid comprises a capsid protein subunit from an AAV of any one of the following serotypes: AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.

99. The recombinant AAV vector of embodiment 97, wherein the protein capsid comprises a capsid protein subunit that has one or more amino acid mutations relative to a capsid protein subunit of any one of the following AAV serotypes: AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.

100. A method of producing a recombinant AAV vector, the method comprising contacting an AAV producer cell with the AAV transfer cassette of any one of embodiments 78-92, the plasmid of embodiment 93, or the nucleic acid of embodiment 93.

101. A recombinant AAV vector produced by the method of embodiment 100.

102. A composition comprising the AAV transfer cassette of any one of embodiments 78-92, the plasmid of embodiment 93, the nucleic acid of embodiment 94 or 95, the cell of embodiment 96, or the recombinant AAV vector of any one of embodiments 97-99 or 101.

10³. A method for treating a subject in need thereof comprising administering to the subject an effective amount of the AAV transfer cassette of any one of embodiments 78-92, the plasmid of embodiment 93, the nucleic acid of embodiment 94 or 95, the cell of embodiment 96, or the recombinant AAV vector of any one of embodiments 97-99 or 101.

104. The method of embodiment 10, wherein the subject has NPC1.

105. The method of embodiment 103 or 104, wherein the subject is a human subject.

What is claimed is:

1. An Adeno-Associated Virus (AAV) transfer cassette comprising, from 5′ to 3′:
a 5′ inverted terminal repeat (ITR);
a chicken beta-actin promoter;
a transgene;
a polyadenylation signal; and
a 3′ ITR;
wherein the transfer cassette comprises an intronic sequence; and
wherein the transgene encodes the NPC1 protein.

2. The AAV transfer cassette of claim 1, wherein the 5′ ITR comprises the sequence of SEQ ID NO: 3.

3. The AAV transfer cassette of claim 1, wherein the 3′ ITR comprises the sequence of SEQ ID NO: 4.

4. The AAV transfer cassette of claim 1, wherein the promoter comprises the sequence of SEQ ID NO: 5.

5. The AAV transfer cassette of claim 1, wherein the intronic sequence is an SV40 intron.

6. The AAV transfer cassette of claim 1, wherein the intronic sequence comprises the sequence of SEQ ID NO: 10.

7. The AAV transfer cassette of claim 1, wherein the NPC1 protein is the human NPC1 protein.

8. The AAV transfer cassette of claim 1, wherein the NPC1 protein comprises the sequence of SEQ ID NO: 1.

9. The AAV transfer cassette of claim 1, wherein the transgene comprises the sequence of SEQ ID NO: 2.

10. The AAV transfer cassette of claim 1, wherein the polyadenylation signal is the SV40 polyadenylation signal.

11. The AAV transfer cassette of claim 1, wherein the polyadenylation signal comprises the sequence of SEQ ID NO: 12.

12. The AAV transfer cassette of claim 1, wherein the cassette comprises an enhancer.

13. The AAV transfer cassette of claim 1, wherein the AAV transfer cassette comprises the sequence of SEQ ID NO: 14.

14. The AAV transfer cassette of claim 1, wherein the AAV transfer cassette comprises the sequence of any one of SEQ ID NO: 15-19.

15. A plasmid comprising the AAV transfer cassette of any one of claims 1-14.

16. A nucleic acid comprising, from 5′ to 3′, a 5′ inverted terminal repeat (ITR); a promoter; a transgene; a polyadenylation signal; and a 3′ ITR; wherein the nucleic acid comprises an intronic sequence; wherein the transgene encodes the amino acid sequence of SEQ ID NO: 1.

17. A cell comprising the AAV transfer cassette of any one of claims 1-14, the plasmid of claim 15, or the nucleic acid of claim 16.

18. A recombinant AAV vector comprising a protein capsid and a nucleic acid encapsidated by the protein capsid:
wherein the nucleic acid comprises the AAV transfer cassette of any one of claims 1-14.

19. The recombinant AAV vector of claim 18, wherein the protein capsid comprises a capsid protein subunit that is from an AAV of any one of the following serotypes: AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.

20. The recombinant AAV vector of claim 18, wherein the protein capsid comprises a capsid protein subunit that has one or more amino acid mutations relative to a capsid protein subunit of any one of the following AAV serotypes: AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.

21. A method of producing a recombinant AAV vector, the method comprising contacting an AAV producer cell with the AAV transfer cassette of any one of claims 1-14, the plasmid of claim 15, or the nucleic acid of claim 16.

22. A recombinant AAV vector produced by the method of claim 21.

23. A composition comprising the AAV transfer cassette of any one of claims 1-14, the plasmid of claim 15, the nucleic acid of claim 16, the cell of claim 17, or the recombinant AAV vector of any one of claims 18-20 or 22.

24. A method for treating a subject in need thereof comprising administering to the subject an effective amount of the AAV transfer cassette of any one of claims 1-14, the plasmid of claim 15, the nucleic acid of claim 16, the cell of claim 17, or the recombinant AAV vector of any one of claims 18-20 or 22.

25. The method of claim 24, wherein the subject has NPC1.

26. The method of claim 24, wherein the subject is a human subject.
